(19) United States
(12) Patent Application Publication
(10) Pub. No.: US 2021/0179519 A1
(43) Pub. Date: Jun. 17, 2021

(54) COMPOUND, SYNTHESIS METHOD OF THE COMPOUND, HARDMASK COMPOSITION, AND METHOD OF FORMING PATTERNS

(71) Applicant: SAMSUNG SDI CO., LTD., Yongin-si (KR)

(72) Inventor: Jaebum LIM, Suwon-si (KR)

(21) Appl. No.: 17/117,324

(22) Filed: Dec. 10, 2020

(30) Foreign Application Priority Data
Dec. 16, 2019 (KR) 10-2019-0168230

(51) Int. Cl. C07C 15/14; C01B 32/184; C07F 5/05; G03F 7/11; G03F 7/09

(52) U.S. Cl. CPC C07C 15/14; C01B 32/184; G03F 7/094; G03F 7/11; C07F 5/05

(57) ABSTRACT

A compound, a synthesis method of the compound, a hardmask composition including the compound, and a method of forming patterns using the hardmask composition, the compound including a condensed or non-condensed polycyclic aromatic core having 40 or more carbon atoms and a plurality of substituents at a periphery of the core, wherein the plurality of substituents are each independently a substituted or unsubstituted C3 to C20 branched alkyl group, a C6 to C30 aryl group substituted with a substituted or unsubstituted C3 to C20 branched alkyl group, a C3 to C30 cycloalkyl group substituted with a substituted or unsubstituted C3 to C20 branched alkyl group, a C3 to C30 heterocyclic group substituted with a substituted or unsubstituted C3 to C20 branched alkyl group, or a combination thereof.

### CROSS-REFERENCE TO RELATED APPLICATION

Korean Patent Application No. 10-2019-0168230, filed on Dec. 16, 2019, in the Korean Intellectual Property Office, and entitled: “Compound, Synthesis Method of the Compound, Hardmask Composition, and Method of Forming Patterns,” is incorporated by reference herein in its entirety.

### BACKGROUND

### 1. Field

Embodiments relate to a compound, a synthesis method of the compound, a hardmask composition including the compound, and a method of forming patterns using the hardmask composition.

### 2. Description of the Related Art

Recently, the semiconductor industry has developed to an ultrafine technique having a pattern of several to several tens nanometer size. Such ultrafine technique use effective lithographic techniques.

One lithographic technique may include providing a material layer on a semiconductor substrate; coating a photoresist layer thereon; exposing and developing the same to provide a photoresist pattern; and etching a material layer using the photoresist pattern as a mask.

### SUMMARY

The embodiments may be realized by providing a compound including a condensed or non-condensed polycyclic aromatic core having 40 or more carbon atoms and a plurality of substituents at a periphery of the core, wherein the plurality of substituents are each independently a substituted or unsubstituted C3 to C20 branched alkyl group, a C6 to C30 aryl group substituted with a substituted or unsubstituted C3 to C20 branched alkyl group, a C3 to C30 cycloalkyl group substituted with a substituted or unsubstituted C3 to C20 branched alkyl group, a C3 to C30 heterocyclic group substituted with a substituted or unsubstituted C3 to C20 branched alkyl group, or a combination thereof.

The plurality of substituents may include a C6 to C30 aryl group substituted with a substituted or unsubstituted C3 to C20 branched alkyl group, and the C6 to C30 aryl group substituted with a substituted or unsubstituted C3 to C20 branched alkyl group may include a phenyl group substituted with at least two substituted or unsubstituted C3 to C20 branched alkyl groups, a naphthyl group substituted with at least two substituted or unsubstituted C3 to C20 branched alkyl groups, an anthracenyl group substituted with at least two substituted or unsubstituted C3 to C20 branched alkyl groups, a phenanthrenyl group substituted with at least two substituted or unsubstituted C3 to C20 branched alkyl groups, a pyrenyl group substituted with at least two substituted or unsubstituted C3 to C20 branched alkyl groups, or a combination thereof.

Two substituents of the plurality of substituents may be bonded to one aromatic ring of the condensed or non-condensed polycyclic aromatic core at ortho positions of the one aromatic ring.

The plurality of substituents may include a C6 to C30 aryl group substituted with a substituted or unsubstituted C3 to C20 branched alkyl group, the C6 to C30 aryl group substituted with a substituted or unsubstituted C3 to C20 branched alkyl group may include a group represented by Chemical Formula 1:

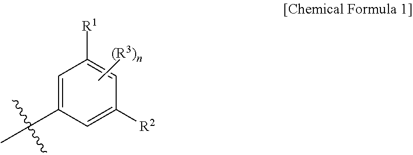

in Chemical Formula 1, R¹ and R² may be independently a substituted or unsubstituted C3 to C20 branched alkyl group, R³ may be independently a substituted or unsubstituted C1 to C30 alkyl group, a substituted or unsubstituted C2 to C30 alkenyl group, a substituted or unsubstituted C2 to C30 alkynyl group, a substituted or unsubstituted C6 to C30 aryl group, a substituted or unsubstituted C3 to C30 heterocyclic group, a halogen, a cyano group, or a combination thereof, n may be an integer of 0 to 3, and

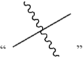

is a linking point with the condensed or non-condensed polycyclic aromatic core.

The substituted or unsubstituted C3 to C20 branched alkyl group may include a substituted or unsubstituted C3 to C20 iso-alkyl group, a substituted or unsubstituted C4 to C20 sec-alkyl group, a substituted or unsubstituted C4 to C20 tert-alkyl group, or a substituted or unsubstituted C5 to C20 neo-alkyl group.

The substituted or unsubstituted C3 to C20 branched alkyl group may include a substituted or unsubstituted iso-propyl group, a substituted or unsubstituted iso-butyl group, a substituted or unsubstituted sec-butyl group, a substituted or unsubstituted tert-butyl group, a substituted or unsubstituted iso-pentyl group, a substituted or unsubstituted sec-pentyl group, a substituted or unsubstituted tert-pentyl group, or a substituted or unsubstituted neo-pentyl group.

The condensed or non-condensed polycyclic aromatic core may include a borazine moiety.

The condensed or non-condensed polycyclic aromatic core may be a condensed polycyclic aromatic ring, and the condensed polycyclic aromatic ring may have a nanographene structure.

A number of the substituents relative to a total number of substitutable positions at the periphery of the condensed polycyclic aromatic core may be greater than or equal to about 10%.

The condensed polycyclic aromatic core may be a particle having a size of about 1 nm to about 20 nm.

The compound may be represented by one of Chemical Formulae 2 to 6:

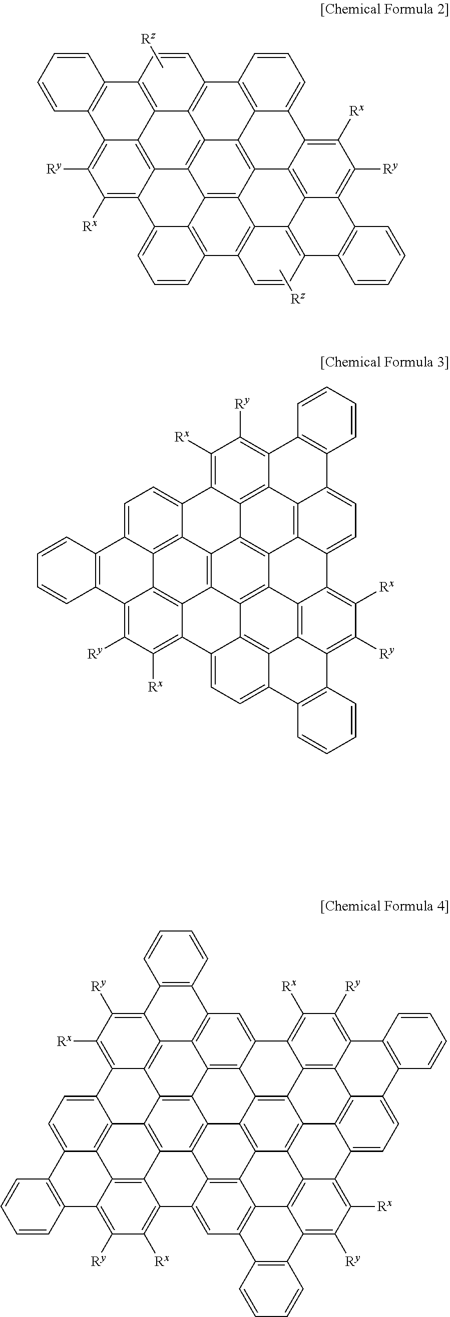

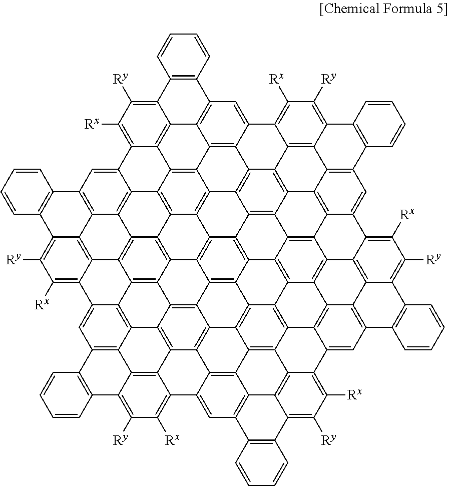

in Chemical Formulae 2 to 6, R^{x} to R^{z} may be independently a substituted or unsubstituted C3 to C20 branched alkyl group, a C6 to C30 aryl group substituted with a substituted or unsubstituted C3 to C20 branched alkyl group, a C3 to C30 cycloalkyl group substituted with a substituted or unsubstituted C3 to C20 branched alkyl group, or a C3 to C30 heterocyclic group substituted with a substituted or unsubstituted C3 to C20 branched alkyl group.

R^{x} and R^{y} may be independently a group represented by Chemical Formula 1:

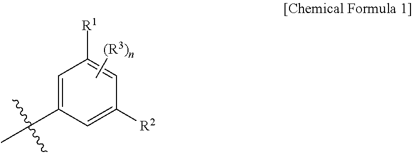

in Chemical Formula 1, R¹ and R² may be independently a substituted or unsubstituted C3 to C20 branched alkyl group, R³ may be a substituted or unsubstituted C1 to C30 alkyl group, a substituted or unsubstituted C2 to C30 alkenyl group, a substituted or unsubstituted C2 to C30 alkynyl group, a substituted or unsubstituted C6 to C30 aryl group, a substituted or unsubstituted C3 to C30 heterocyclic group, or a combination thereof, n may be an integer of 0 to 3, and

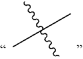

is a linking point with the nanographene structure.

R^{z} may be a substituted or unsubstituted C3 to C20 branched alkyl group.

The condensed or non-condensed polycyclic aromatic core may be a non-condensed polycyclic aromatic core, and the compound may be represented by one of Chemical Formulae 7 to 11:

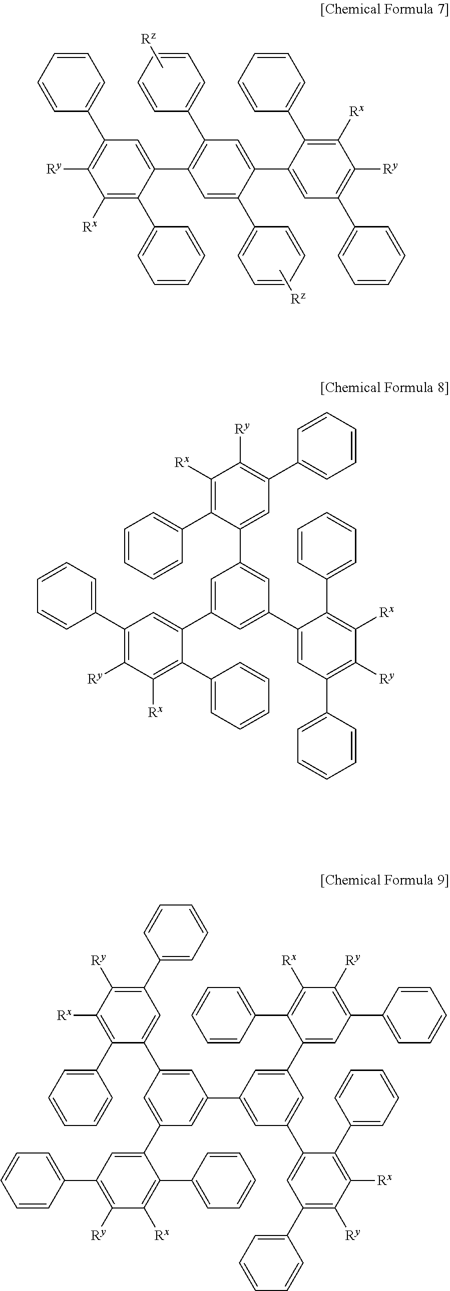

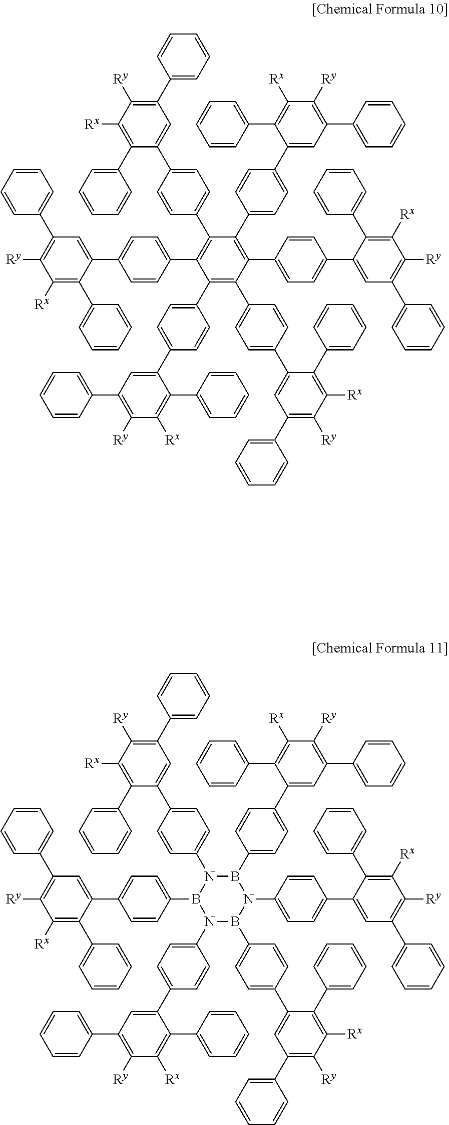

in Chemical Formulae 7 to 11, R^{x} to R^{z} may be independently a substituted or unsubstituted C3 to C20 branched alkyl group, a C6 to C30 aryl group substituted with a substituted or unsubstituted C3 to C20 branched alkyl group, a C3 to C30 cycloalkyl group substituted with a substituted or unsubstituted C3 to C20 branched alkyl group, or a C3 to C30 heterocyclic group substituted with a substituted or unsubstituted C3 to C20 branched alkyl group.

R^{x} and R^{y} may be independently a group represented by Chemical Formula 1:

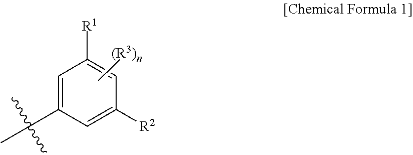

in Chemical Formula 1, R¹ and R² may be independently a substituted or unsubstituted C3 to C20 branched alkyl group, R³ may be a substituted or unsubstituted C1 to C30 alkyl group, a substituted or unsubstituted C2 to C30 alkenyl group, a substituted or unsubstituted C2 to C30 alkynyl group, a substituted or unsubstituted C6 to C30 aryl group, a substituted or unsubstituted C3 to C30 heterocyclic group, or a combination thereof, n may be an integer of 0 to 3, and

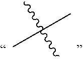

is a linking point with the non-condensed polycyclic aromatic core.

The embodiments may be realized by providing a method of synthesizing a compound, the method including preparing a compound represented by Chemical Formula 12, and reacting the compound represented by Chemical Formula 12 with an aromatic compound having at least two ethynyl groups to obtain a polycyclic aromatic compound,

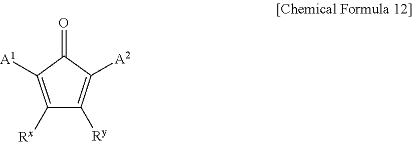

wherein, in Chemical Formula 12, A¹ and A² are independently a substituted or unsubstituted phenyl group, and R^{x} and R^{y} are independently a substituted or unsubstituted C3 to C20 branched alkyl group, a C6 to C30 aryl group substituted with a substituted or unsubstituted C3 to C20 branched alkyl group, a C3 to C30 cycloalkyl group substituted with a substituted or unsubstituted C3 to C20 branched alkyl group, or a C3 to C30 heterocyclic group substituted with a substituted or unsubstituted C3 to C20 branched alkyl group.

The aromatic compound having at least two ethynyl groups may include a single aromatic ring or a non-condensed aromatic ring structure in which two or more aromatic rings are linked by a single bond.

The method may further include forming a condensed polycyclic aromatic ring having 40 or more carbon atoms by cyclodehydrogenation of the polycyclic aromatic compound, wherein the condensed polycyclic aromatic ring having 40 or more carbon atoms has a nanographene structure.

The embodiments may be realized by providing a hardmask composition including the compound according to an embodiment; and a solvent.

The embodiments may be realized by providing a method of forming patterns, the method including applying the hardmask composition according to an embodiment on a material layer and heat treating the hardmask composition to form a hardmask layer, forming a photoresist layer on the hardmask layer, exposing and developing the photoresist layer to form a photoresist pattern, selectively removing the hardmask layer using the photoresist pattern to expose a portion of the material layer, and etching an exposed portion of the material layer.

### BRIEF DESCRIPTION OF THE DRAWINGS

Features will be apparent to those of skill in the art by describing in detail exemplary embodiments with reference to the attached drawings in which:

FIGS. 1 and 2 are ¹HNMR spectra (¹H nuclear magnetic resonance spectra) of the compound represented by Chemical Formula 1a and the compound represented by Chemical Formula 1b, respectively,

FIG. 3 is a MALDI-TOF mass spectrum of the compound represented by Chemical Formula 1c,

FIG. 4 shows gel permeation chromatography (GPC) spectra of 1,3,5-triethynylbenzene (TEB), a compound represented by Chemical Formula 1b and a compound represented by Chemical Formula 1c,

FIG. 5 is a MALDI-T of mass spectrum of the compound represented by Chemical Formula 1d,

FIG. 6 shows gel permeation chromatography (GPC) spectra of 3,3′,5′,5′-tetraethynylbiphenyl (TEBP), a compound represented by Chemical Formula 1b, and a compound represented by Chemical Formula 1d,

FIG. 7 is a MALDI-TOF mass spectrum of the compound represented by Chemical Formula 1e,

FIG. 8 shows gel permeation chromatography (GPC) spectra of 1,3,5-triethynylbenzene (TEB), a compound represented by Chemical Formula 1b, a compound represented by Chemical Formula 1c, and a compound represented by Chemical Formula 1e,

FIG. 9 is a MALDI-TOF mass spectrum of the compound represented by Chemical Formula 1f, and

FIG. 10 shows gel permeation chromatography (GPC) spectra of 3,3′,5′,5′-tetraethynylbiphenyl (TEBP), compound represented by Chemical Formula 1b, a compound represented by Chemical Formula 1d, and a compound represented by Chemical Formula 1f.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout.

Hereinafter, as used herein, when a definition is not otherwise provided, “substituted” refers to replacement of a hydrogen atom of a compound by a substituent selected from deuterium, a halogen atom (F, Br, Cl, or I), a hydroxy group, a nitro group, a cyano group, an amino group, an azido group, an amidino group, a hydrazino group, a hydrazono group, a carbonyl group, a carbamyl group, mercapto group, an ester group, a carboxyl group or a salt thereof, a sulfonic acid group or a salt thereof, a phosphoric acid group or a salt thereof, a C1 to C30 alkyl group, a C2 to C30 alkenyl group, a C2 to C30 alkynyl group, a C6 to C30 aryl group, a C7 to C30 arylalkyl group, a C1 to C30 alkoxy group, a C1 to C30 alkylthiol group C1 to C20 heteroalkyl group, a C3 to C20 heteroarylalkyl group, a C3 to C30 cycloalkyl group, a C3 to C15 cycloalkenyl group, a C6 to C15 cycloalkynyl group, a C2 to C30 heterocyclic group, and a combination thereof.

In addition, adjacent two substituents of the halogen atom (F, Br, Cl, or I), hydroxy group, nitro group, cyano group, amino group, azido group, amidino group, hydrazino group, hydrazono group, carbonyl group, carbamyl group, mercapto group, ester group, carboxyl group or salt thereof, sulfonic acid group or salt thereof, phosphoric acid group or salt thereof, C1 to C30 alkyl group, C2 to C30 alkenyl group, C2 to C30 alkynyl group, C6 to C30 aryl group, C7 to C30 arylalkyl group, C1 to C30 alkoxy group, C1 to C30 alkylthiol group, C1 to C20 heteroalkyl group, C3 to C20 heteroarylalkyl group, C3 to C30 cycloalkyl group, C3 to C15 cycloalkenyl group, C6 to C15 cycloalkynyl group, and C2 to C30 heterocyclic group may be fused to form a ring. For example, the substituted C6 to C30 aryl group may be fused with another adjacent substituted C6 to C30 aryl group to form a substituted or unsubstituted fluorene ring.

As used herein, when a definition is not otherwise provided, “hetero” refers to one including 1 to 3 heteroatoms selected from N, O, S, Se, and P.

As used herein, “aryl group” refers to a group including at least one hydrocarbon aromatic moiety, and includes hydrocarbon aromatic moieties linked by a single bond and hydrocarbon aromatic moieties fused directly or indirectly to provide a non-aromatic fused ring. The aryl group may include a monocyclic, polycyclic or fused polycyclic (i.e., rings sharing adjacent pairs of carbon atoms) functional group.

As used herein, “heterocyclic group” is a concept including a heteroaryl group, and may include at least one hetero atom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

More specifically, the substituted or unsubstituted aryl group may be a substituted or unsubstituted phenyl group, a substituted or unsubstituted naphthyl group, a substituted or unsubstituted anthracenyl group, a substituted or unsubstituted phenanthryl group, a substituted or unsubstituted naphthacenyl group, a substituted or unsubstituted pyrenyl group, a substituted or unsubstituted biphenyl group, a substituted or unsubstituted terphenyl group, a substituted or unsubstituted quaterphenyl group, a substituted or unsubstituted chrysenyl group, a substituted or unsubstituted triphenylenyl group, a substituted or unsubstituted perylenyl group, a substituted or unsubstituted indenyl group, a substituted or unsubstituted fluorenyl group, a combination thereof, or a combined fused ring of the foregoing groups.

More specifically, the substituted or unsubstituted heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridinyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted dibenzofuranyl group, a substituted or unsubstituted dibenzothiophenyl group, a substituted or unsubstituted carbazolyl group, pyridoindolyl group, benzopyridooxazinyl group, benzopyridothiazinyl group, 9,9-dimethyl-9,10 dihydroacridinyl group, a combination thereof, or a combined fused ring of the foregoing groups. In one example, the heterocyclic group or the heteroaryl group may be a pyridyl group, a carbazolyl group, or a pyridoindolyl group.

As used herein, “aromatic ring” refers to a form in which a non-condensed aromatic ring, a condensed aromatic ring, and aromatic rings are linked by a single bond, a form in which each ring condensed on two sides that are not parallel to each other on a benzene ring is fused, a form in which each ring condensed on two sides that are not parallel to each other on a benzene ring is linked by a single bond or a double bond, or a combination thereof.

In the present specification, an ethynyl group refers to a functional group in which a carbon-carbon triple bond (—C≡CH) is located at the terminal end of the compound.

Hereinafter, a compound according to an embodiment is described.

The compound according to an embodiment may include a polycyclic aromatic core including a plurality of aromatic rings. In an implementation, an aromatic ring at an end (e.g., outer edge or exterior) of the polycyclic aromatic core may be substituted with one or more substituents.

The polycyclic aromatic core may be a condensed or non-condensed polycyclic aromatic core having 40 or more carbon atoms, e.g., a condensed or non-condensed polycyclic aromatic core having 48 or more, 50 or more, or 54 or more carbon atoms, e.g., a condensed or non-condensed polycyclic aromatic core having 250 or fewer carbon atoms, 230 or fewer carbon atoms, 210 or fewer carbon atoms, 200 or fewer carbon atoms, 180 or fewer carbon atoms, or 150 or fewer carbon atoms. In an implementation, the polycyclic aromatic core may be a condensed polycyclic aromatic core in which a plurality of aromatic rings are condensed, and may have, e.g., a honeycomb-shaped condensed structure. In an implementation, the polycyclic aromatic core may be a non-condensed polycyclic aromatic core in which a plurality of aromatic rings are linked by single bonds. In an implementation, it may have a structure in which a plurality of aromatic rings extend radially around or from one or two (e.g., central) aromatic rings. The condensed polycyclic aromatic core and the non-condensed polycyclic aromatic core will be described in greater detail below.

In an implementation, the substituent may be bonded to an aromatic ring at terminal end (e.g., edge, periphery, or outer area) of the polycyclic aromatic core. In an implementation, the substituent may include a bulky group capable of providing a steric hindrance to the compound. In an implementation, the substituent may include a substituted or unsubstituted C3 to C20 branched hydrocarbon, e.g., a substituted or unsubstituted C3 to C20 branched alkyl group. When the compound includes a substituted or unsubstituted C3 to C20 branched alkyl group, a solubility of the compound in an organic solvent may be increased, and it may be effectively applied to a solution process such as spin-on coating.

In an implementation, the substituent may include a substituted or unsubstituted C3 to C20 branched alkyl group, an aromatic or aliphatic cyclic group substituted with a substituted or unsubstituted C3 to C20 branched alkyl group, or a combination thereof. As used herein, the term “or” is not an exclusive term, e.g., “A or B” would include A, B, or A and B.

In an implementation, the substituent may include an aromatic or aliphatic cyclic group substituted with a substituted or unsubstituted C3 to C20 branched alkyl group, e.g., a C6 to C30 aryl group substituted with a substituted or unsubstituted C3 to C20 branched alkyl group, a C3 to C30 cycloalkyl group substituted with a substituted or unsubstituted C3 to C20 branched alkyl group, a C3 to C30 heterocyclic group substituted with a substituted or unsubstituted C3 to C20 branched alkyl group, or a combination thereof. In an implementation, by including the aromatic or aliphatic cyclic group substituted with the substituted or unsubstituted C3 to C20 branched alkyl group as a substituent, steric hindrance may be further caused by rotation of the substituent at the edge of the compound, thereby further increasing a solubility of the compound in an organic solvent, and being more effectively applied to solution processes such as spin-on coating.

The aforementioned compound may not further include a hydroxy group, an amino group, a mercapto group, a substituted or unsubstituted C1 to C30 alkoxy group, a substituted or unsubstituted C1 to C30 alkylamino group and/or a substituted or unsubstituted a C1 to C30 thioalkoxy group in addition to the aforementioned substituent at the periphery of the polycyclic aromatic core. Accordingly, the aforementioned substituent may help improve the solubility of the compound while increasing a carbon content, and a hard layer may be formed after curing, thereby providing a layer having high etch resistance, e.g., a layer having higher etch resistance to CFx etching gas.

In an implementation, two of the plurality of substituents may be bonded to one (e.g., a same) aromatic ring. In an implementation, two of the plurality of substituents may be bonded to positions adjacent to one another on the aromatic ring. In an implementation, two of the plurality of substituents may be bonded to the one aromatic ring in an ortho arrangement. By further causing steric hindrance between a plurality of substituents at the periphery of the compound, a solubility in the solvent may be further improved and thereby interaction and/or stacking between compounds may be reduced or prevented.

In an implementation, the substituted or unsubstituted C3 to C20 branched alkyl group may include, e.g., a substituted or unsubstituted C3 to C20 iso-alkyl group, a substituted or unsubstituted C4 to C20 sec-alkyl group, a substituted or unsubstituted C4 to C20 tert-alkyl group, or a substituted or unsubstituted C5 to C20 neo-alkyl group. In an implementation, the substituted or unsubstituted C3 to C20 branched alkyl group may include, e.g., a substituted or unsubstituted C4 to C20 tert-alkyl group.

In an implementation, the substituted or unsubstituted C3 to C20 branched alkyl group may include, e.g., a substituted or unsubstituted iso-propyl group, a substituted or unsubstituted iso-butyl group, a substituted or unsubstituted sec-butyl group, a substituted or unsubstituted tert-butyl group, a substituted or unsubstituted iso-pentyl group, a substituted or unsubstituted sec-pentyl group, a substituted or unsubstituted tert-pentyl group, or a substituted or unsubstituted neo-pentyl group. In an implementation, the substituted or unsubstituted C3 to C20 branched alkyl group may include, e.g., a substituted or unsubstituted tert-butyl group. In an implementation, the solubility of the compound may be further improved.

In an implementation, the C6 to C30 aryl group substituted with a substituted or unsubstituted C3 to C20 branched alkyl group may include, e.g., a phenyl group substituted with a substituted or unsubstituted C3 to C20 branched alkyl group, a naphthyl group substituted with a substituted or unsubstituted C3 to C20 branched alkyl group, an anthracenyl group substituted with a substituted or unsubstituted C3 to C20 branched alkyl group, a phenanthrenyl group substituted with a substituted or unsubstituted C3 to C20 branched alkyl group, or a pyrenyl group substituted with a substituted or unsubstituted C3 to C20 branched alkyl group. In an implementation, the C6 to C30 aryl group substituted with a substituted or unsubstituted C3 to C20 branched alkyl group may include, e.g., a phenyl group substituted with a substituted or unsubstituted C3 to C20 branched alkyl group. The substituted or unsubstituted C3 to C20 branched alkyl group may be the same as described above.

In an implementation, the C6 to C30 aryl group substituted with a substituted or unsubstituted C3 to C20 branched alkyl group may include, e.g., a C6 to C30 aryl group substituted with at least two substituted or unsubstituted C3 to C20 branched alkyl groups.

In an implementation, the C6 to C30 aryl group substituted with a substituted or unsubstituted C3 to C20 branched alkyl group may include, e.g., a phenyl group substituted with at least two substituted or unsubstituted C3 to C20 branched alkyl groups, a naphthyl group substituted with at least two substituted or unsubstituted C3 to C20 branched alkyl groups, an anthracenyl group substituted with at least two substituted or unsubstituted C3 to C20 branched alkyl groups, a phenanthrenyl group substituted with at least two substituted or unsubstituted C3 to C20 branched alkyl groups, a pyrenyl group substituted with at least two substituted or unsubstituted C3 to C20 branched alkyl groups, or a combination thereof. In an implementation, the C6 to C30 aryl group substituted with a substituted or unsubstituted C3 to C20 branched alkyl group may include, e.g., a phenyl group substituted with at least two substituted or unsubstituted C3 to C20 branched alkyl groups. The substituted or unsubstituted C3 to C20 branched alkyl group may be the same as described above.

In an implementation, the C6 to C30 aryl group substituted with a substituted or unsubstituted C3 to C20 branched alkyl group may include, e.g., a C6 to C30 aryl group substituted with at least two substituted or unsubstituted C3 to C20 branched alkyl groups.

In an implementation, the C6 to C30 aryl group substituted with a substituted or unsubstituted C3 to C20 branched alkyl group may include, e.g., a phenyl group substituted with two (e.g., only two) substituted or unsubstituted C3 to C20 branched alkyl groups, a naphthyl group substituted with two substituted or unsubstituted C3 to C20 branched alkyl groups, an anthracenyl group substituted with two substituted or unsubstituted C3 to C20 branched alkyl groups, a phenanthrenyl group substituted with two substituted or unsubstituted C3 to C20 branched alkyl groups, or a pyrenyl group substituted with two substituted or unsubstituted C3 to C20 branched alkyl groups. In an implementation, the C6 to C30 aryl group substituted with a substituted or unsubstituted C3 to C20 branched alkyl group may include, e.g., a phenyl group substituted with two substituted or unsubstituted C3 to C20 branched alkyl groups. In an implementation, the two substituted or unsubstituted C3 to C20 branched alkyl groups may be present at a meta position to each other. The substituted or unsubstituted C3 to C20 branched alkyl group may be the same as described above.

In an implementation, the C6 to C30 aryl group substituted with a substituted or unsubstituted C3 to C20 branched alkyl group may include, e.g., a phenyl group substituted with two substituted or unsubstituted C3 to C20 branched alkyl groups. In an implementation, the C6 to C30 aryl group substituted with a substituted or unsubstituted C3 to C20 branched alkyl group may include, e.g., a group represented by Chemical Formula 1.

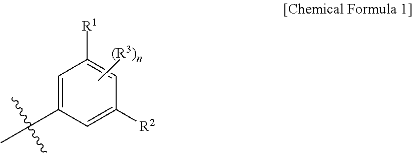

In Chemical Formula 1,

R¹ and R² may each independently be or include, e.g., a substituted or unsubstituted C3 to C20 branched alkyl group,

R³ may be or may include, e.g., a substituted or unsubstituted C1 to C30 alkyl group, a substituted or unsubstituted C2 to C30 alkenyl group, a substituted or unsubstituted C2 to C30 alkynyl group, a substituted or unsubstituted C6 to C30 aryl group, a substituted or unsubstituted C3 to C30 heterocyclic group, a halogen, a cyano group, or a combination thereof,

n may be an integer of, e.g., 0 to 3, and

“ ” is a linking point with the condensed or non-condensed polycyclic aromatic core.

In the definition of R¹ and R² in Chemical Formula 1, the substituted or unsubstituted C3 to C20 branched alkyl group may be the same as described above.

In an implementation, the compound may include two or more substituents represented by Chemical Formula 1, e.g., 4 or more, 6 or more, 8 or more, 10 or more, or 12 or more. In an implementation, the compound may include two or more substituents represented by Chemical Formula 1, e.g., 2 to 30, 4 to 30, 6 to 30, 8 to 30, 10 to 30, or 12 to 30.

The compound may include a plurality of groups represented by Chemical Formula 1 at the periphery of the compound, e.g., outside of the core, and a carbon content of the compound may increase to form a hard layer, thereby providing higher etch resistance. In addition, by causing steric hindrance between the adjacent substituents to an appropriate level, distortion of the compound may be induced to an appropriate level, and thus, solubility of the compound in a solvent may be improved.

In an implementation, R¹ and R² may be the same as or different from each other. In an implementation, R¹ and R² may be the same as each other. In an implementation, both R¹ and R² may be a substituted or unsubstituted C4 to C20 tert alkyl group, e.g., both R¹ and R² may be a substituted or unsubstituted tert-butyl group.

In an implementation, n may be 0 or 1. In an implementation, n may be 0.

In an implementation, the C3 to C30 cycloalkyl group substituted with a substituted or unsubstituted C3 to C20 branched alkyl group may include, e.g., a substituted or unsubstituted cyclopentyl group, a substituted or unsubstituted cyclohexyl group, or a substituted or unsubstituted cycloheptyl group. The substituted or unsubstituted C3 to C20 branched alkyl group may the same as described above.

In an implementation, the C3 to C30 cycloalkyl group substituted with a substituted or unsubstituted C3 to C20 branched alkyl group may be, e.g., a C6 to C30 cycloalkyl group substituted with at least two substituted or unsubstituted C3 to C20 branched alkyl groups.

In an implementation, the C3 to C30 cycloalkyl group substituted with a substituted or unsubstituted C3 to C20 branched alkyl group may be, e.g., a cyclopentyl group substituted with at least two substituted or unsubstituted C3 to C20 branched alkyl groups, a cyclohexyl group substituted with at least two substituted or unsubstituted C3 to C20 branched alkyl groups, or a heptyl group substituted with at least two substituted or unsubstituted C3 to C20 branched alkyl groups. The substituted or unsubstituted C3 to C20 branched alkyl group may be the same as described above.

In an implementation, the C3 to C30 heterocyclic group substituted with a substituted or unsubstituted C3 to C20 branched alkyl group may include, e.g., a pyridyl group substituted with a substituted or unsubstituted C3 to C20 branched alkyl group, a pyrimidinyl group substituted with a substituted or unsubstituted C3 to C20 branched alkyl group, a pyridazinyl group substituted with a substituted or unsubstituted C3 to C20 branched alkyl group, or a pyrazinyl group substituted with a substituted or unsubstituted C3 to C20 branched alkyl group. The substituted or unsubstituted C3 to C20 branched alkyl group may be the same as described above.

In an implementation, the C3 to C30 heterocyclic group substituted with a substituted or unsubstituted C3 to C20 branched alkyl group may include, e.g., a C3 to C30 heterocyclic group substituted with at least two substituted or unsubstituted C3 to C20 branched alkyl groups.

In an implementation, the C3 to C30 heterocyclic group substituted with a substituted or unsubstituted C3 to C20 branched alkyl group may include, e.g., a pyridyl group substituted with at least two substituted or unsubstituted C3 to C20 branched alkyl groups, a pyrimidinyl group substituted with at least two substituted or unsubstituted C3 to C20 branched alkyl groups, a pyridazinyl group substituted with at least two substituted or unsubstituted C3 to C20 branched alkyl groups, or a pyrazinyl group substituted with at least two substituted or unsubstituted C3 to C20 branched alkyl groups. The substituted or unsubstituted C3 to C20 branched alkyl group may be the same as described above.

As described above, the compound may have a high solubility in a solvent by including the aforementioned plurality of substituents at the periphery of the condensed or non-condensed polycyclic aromatic core, and at the same time, the compound may include the condensed or non-condensed polycyclic aromatic core having 40 or more carbon atoms, a carbon content thereof may be further increased, a hard layer may be formed, and thus higher etch resistance may be imparted.

The condensed or non-condensed polycyclic aromatic core may not be made of all carbon and/or hydrogen atoms, and some atoms other than carbon and hydrogen may be mixed or included therein. In an implementation, the atoms other than carbon and hydrogen may include, e.g., a boron atom, an oxygen atom, a nitrogen atom, a phosphorus atom, or a sulfur atom. In an implementation, the condensed or non-condensed polycyclic aromatic core may include a borazine moiety (e.g., a moiety derived from borazine).

The condensed or non-condensed polycyclic aromatic core may be a condensed polycyclic aromatic ring, a harder layer may be formed, and thus higher etch resistance may be imparted. In an implementation, the condensed or non-condensed polycyclic aromatic core may be a condensed polycyclic aromatic ring having, e.g., 40 or more carbon atoms, 48 or more carbon atoms, 50 or more carbon atoms, or 54 or more carbon atoms, and, e.g., 250 or fewer carbon atoms, 230 or fewer carbon atoms, 210 or fewer carbon atoms, 200 or fewer carbon atoms, 180 or fewer carbon atoms, or 150 or fewer carbon atoms.

In an implementation, the condensed polycyclic aromatic ring may be nanographene (e.g., may have a nanographene structure). The nanographene may have a polycyclic aromatic ring having a size of several A to hundreds of nm by covalently linking a plurality of carbon atoms to each other, wherein the carbon atoms linked each other through the covalent bond forms a 6-membered ring as a basic repeating unit, and may further include a 5-membered ring and/or a 7-membered ring. The nanographene of the core may form a honeycomb-shaped two-dimensional planar structure in which a plurality of hexagons are linked side by side (e.g., fused) when the covalently-bonded carbon atoms (in general, a sp² bond) are conjugated.

A method of preparing the nanographene may include, e.g., a method (top-down) of physically/chemically peeling off graphite or a method (bottom-up) of synthesizing nanographene from an organic material such as small molecules. In an implementation, the method (bottom-up) of synthesizing nanographene from an organic material such as small molecules may easily form the nanographene to have a desired structure and size. In an implementation, the condensed polycyclic aromatic ring may include nanographene synthesized from an organic material in the method (bottom-up).

The compound may include the nanographene as a core and thus may include sp² carbon in a larger amount than in other amorphous carbon films. The sp² carbon is conjugated, bonding electrons are delocalized, and sp² carbon-sp² carbon bonding energy may be larger than sp^{a} carbon-sp^{a} carbon bonding energy. The carbon-carbon bonding energy constituting the nanographene of the core may become larger, and breakage of the nanographene in the core may be more difficult.

In an implementation, the compound may include the nanographene as a core and more the sp² carbon than the sp^{a} carbon and accordingly, may have a high carbon vs. hydrogen ratio (C/H ratio) and thus high etch resistance against dry etching gas.

In an implementation, a ratio of a number of substituents to a total number of substitutable positions at the periphery of the condensed polycyclic aromatic core may be greater than or equal to about 10%, e.g., about 10% to about 55%, about 20 to about 50%, or about 25% to about 43%. For example, at least about 10% of the substitutable positions at the periphery of the condensed polycyclic aromatic core may include a substituent thereon. In an implementation, solubility of the compound having the condensed polycyclic aromatic core for a solvent may be improved.

In an implementation, the condensed polycyclic aromatic core may be a particle having a size of about 1 nm to about 20 nm, e.g., about 1 nm to about 15 nm, about 1 nm to about 10 nm, or about 1 nm to about 5 nm. When the condensed polycyclic aromatic core has a spherical shape, the “size” denotes an average particle diameter of the condensed polycyclic aromatic core. When the condensed polycyclic aromatic core has a sheet-shaped structure, the “size” denotes a two dimensional planar diameter, and when the condensed polycyclic aromatic core is oval, the “size” may denote a long-axis diameter. As the compound has the condensed polycyclic aromatic core having a size within the aforementioned range, when a lower substrate (or a film) has a step difference or is patterned, the compound may form a layer having more excellent gap-fill and planarization characteristics.

In an implementation, the condensed or non-condensed polycyclic aromatic core may be a condensed polycyclic aromatic ring, and the compound may be represented by one of Chemical Formulae 2 to 6.

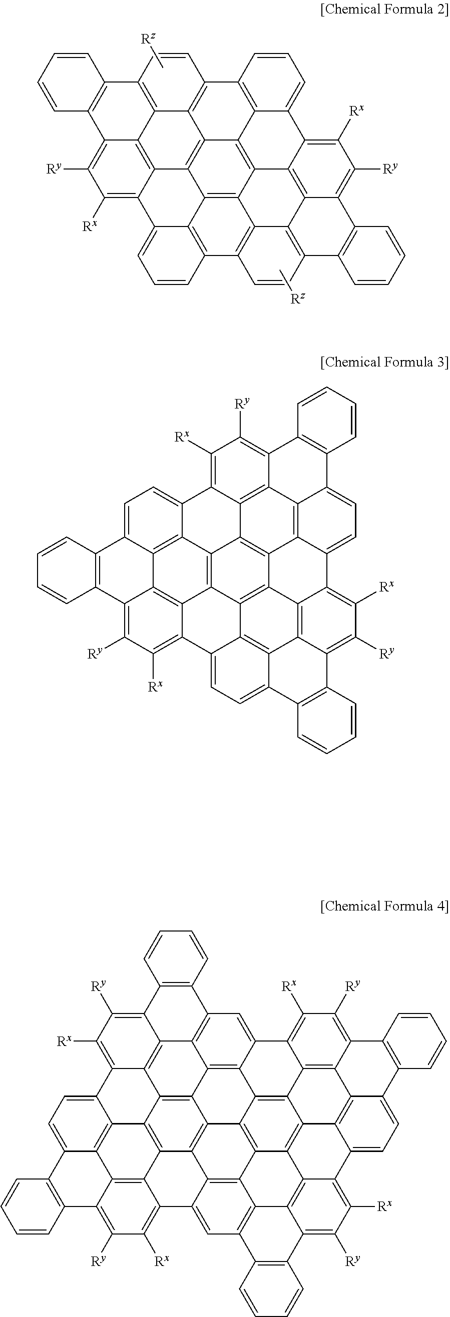

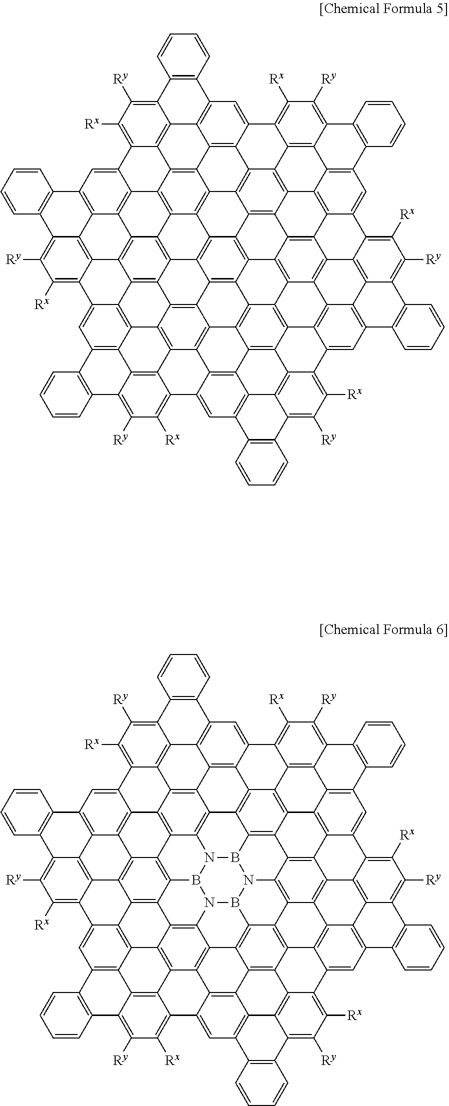

In Chemical Formulae 2 to 6, R^{x} to R^{z} may each independently be or include, e.g., a substituted or unsubstituted C3 to C20 branched alkyl group, a C6 to C30 aryl group substituted with a substituted or unsubstituted C3 to C20 branched alkyl group, a C3 to C30 cycloalkyl group substituted with a substituted or unsubstituted C3 to C20 branched alkyl group, or a C3 to C30 heterocyclic group substituted with a substituted or unsubstituted C3 to C20 branched alkyl group. The substituted or unsubstituted C3 to C20 branched alkyl group, the C6 to C30 aryl group substituted with a substituted or unsubstituted C3 to C20 branched alkyl group, the C3 to C30 cycloalkyl group substituted with a substituted or unsubstituted C3 to C20 branched alkyl group, and the C3 to C30 heterocyclic group substituted with a one substituted or unsubstituted C3 to C20 branched alkyl group may be the same as described above.

In an implementation, in Chemical Formulae 2 to 6, at least one of R^{x} and R^{y} may be a C6 to C30 aryl group substituted with a substituted or unsubstituted C3 to C20 branched alkyl group. In an implementation, R^{x} and R^{y} may each independently be or include, e.g., a C6 to C30 aryl group substituted with a substituted or unsubstituted C3 to C20 branched alkyl group. The C6 to C30 aryl group substituted with the substituted or unsubstituted C3 to C20 branched alkyl group may be the same as described above.

In an implementation, in Chemical Formulas 2 to 6, R^{x} and R^{y} may each independently be, e.g., a group represented by Chemical Formula 1.

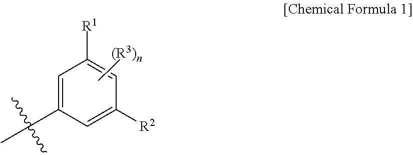

In Chemical Formula 1, R¹ to R³, n and

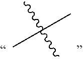

may be the same as described above.

In an implementation, in Chemical Formulae 2 to 6, R^{x} and R^{y} may be the same as or different from each other. In an implementation, in Chemical Formulae 2 to 6, R^{x} and R^{y} may be the same as each other.

In an implementation, in Chemical Formulae 2 to 6, R^{z} may be or may include, e.g., a substituted or unsubstituted C3 to C20 branched alkyl group. The substituted or unsubstituted C3 to C20 branched alkyl group may be the same as described above.

In an implementation, in Chemical Formulae 2 to 6, R^{z} may be a substituted or unsubstituted C4 to C20 tert-alkyl group, e.g., a substituted or unsubstituted tert-butyl group.

In an implementation, the condensed or non-condensed polycyclic aromatic core may be a non-condensed polycyclic aromatic ring, e.g., a non-condensed polycyclic aromatic ring having 40 or more carbon atoms, 48 or more carbon atoms, 50 or more carbon atoms, or 54 or more carbon atoms, and 250 or fewer carbon atoms, 230 or fewer carbon atoms, 210 or fewer carbon atoms, 200 or fewer carbon atoms, 180 or fewer carbon atoms, or 150 or fewer carbon atoms.

In an implementation, the condensed or non-condensed polycyclic aromatic core may be a non-condensed polycyclic aromatic ring, and the compound may be represented by one of Chemical Formulae 7 to 11.

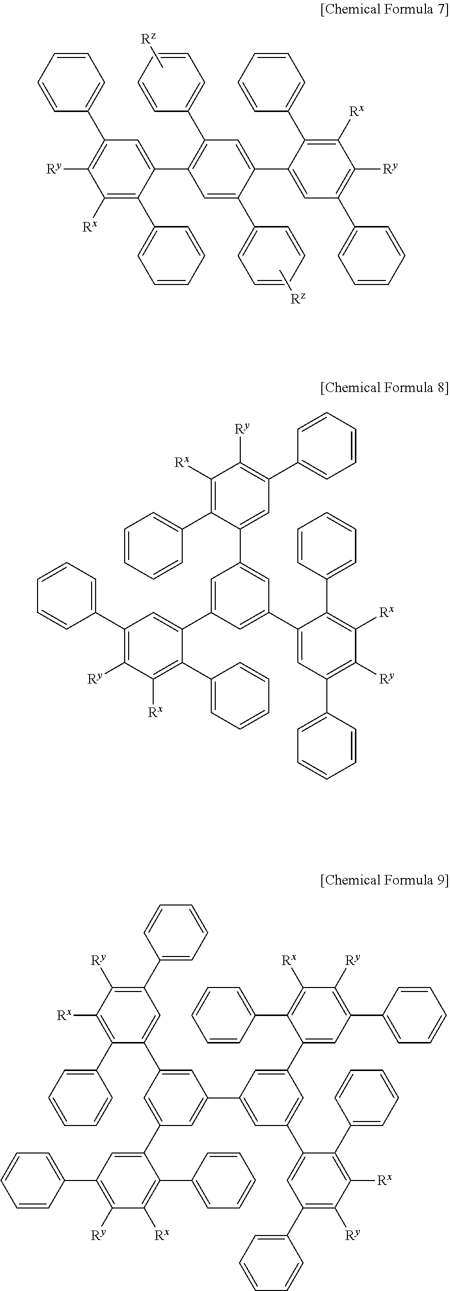

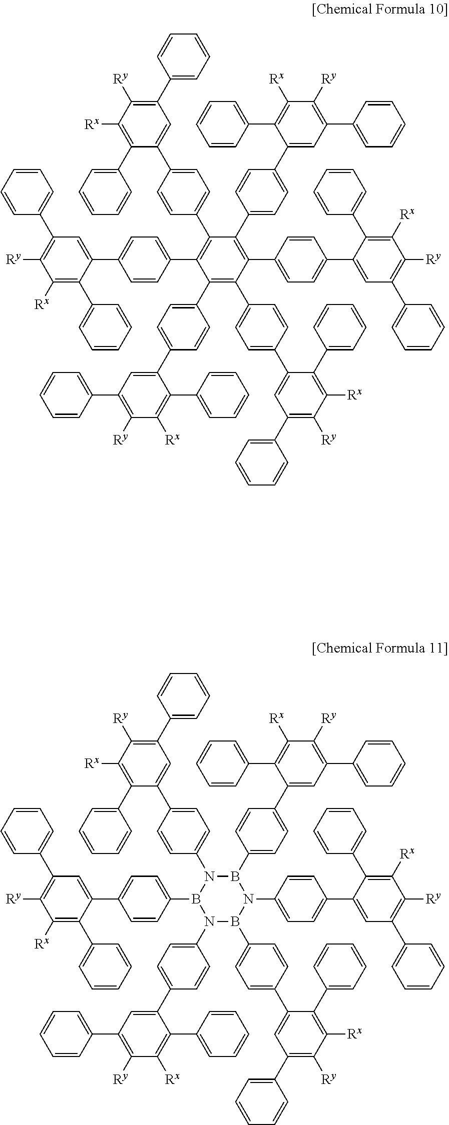

In Chemical Formulae 7 to 11, R^{x} to R^{z} may each independently be or include, e.g., a substituted or unsubstituted C3 to C20 branched alkyl group, a C6 to C30 aryl group substituted with a substituted or unsubstituted C3 to C20 branched alkyl group, a C3 to C30 cycloalkyl group substituted with a substituted or unsubstituted C3 to C20 branched alkyl group, or a C3 to C30 heterocyclic group substituted with a substituted or unsubstituted C3 to C20 branched alkyl group. The substituted or unsubstituted C3 to C20 branched alkyl group, the C6 to C30 aryl group substituted with a substituted or unsubstituted C3 to C20 branched alkyl group, the C3 to C30 cycloalkyl group substituted with a substituted or unsubstituted C3 to C20 branched alkyl group, or the C3 to C30 heterocyclic group substituted with a substituted or unsubstituted C3 to C20 branched alkyl group may be the same as described above.

In an implementation, in Chemical Formulae 7 to 11, at least one of R^{x} and R^{y} may be a C6 to C30 aryl group substituted with a substituted or unsubstituted C3 to C20 branched alkyl group. In an implementation, R^{x} and R^{y} may each independently be or include, e.g., a C6 to C30 aryl group substituted with a substituted or unsubstituted C3 to C20 branched alkyl group. The C6 to C30 aryl group substituted with the substituted or unsubstituted C3 to C20 branched alkyl group may be the same as described above.

In an implementation, in Chemical Formulae 7 to 11, R^{x} and R^{y} may each independently be, e.g., a group represented by Chemical Formula 1.

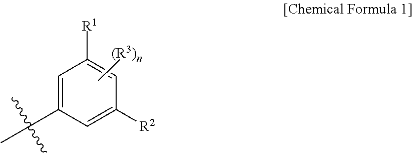

In Chemical Formula 1, R¹ to R³, n and

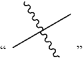

may be the same as described above.

In an implementation, in Chemical Formulae 7 to 11, R^{x} and R^{y} may be the same as or different from each other. In an implementation, in Chemical Formulae 7 to 11, R^{x} and R^{y} may be the same as each other.

In an implementation, in Chemical Formulae 7 to 11, R^{z} may be or may include, e.g., a substituted or unsubstituted C3 to C20 branched alkyl group. The substituted or unsubstituted C3 to C20 branched alkyl group may be the same as described above.

In an implementation, in Chemical Formulae 7 to 11, R^{z} may be or may include, e.g., a substituted or unsubstituted C4 to C20 tert-alkyl group. In an implementation, in Chemical Formulae 7 to 11, R^{z} may be or may include, e.g., a substituted or unsubstituted tert-butyl group.

In an implementation, a molecular weight of the aforementioned compound may be, e.g., about 500 to about 30,000, about 700 to about 10,000, or about 1,000 to about 5,000.

When the compound having the molecular weight is used, a uniform thin film may be formed without generation of a pin-hole and a void or degradation of thickness distribution during a baking process, and excellent gap-fill and planarization characteristics may also be obtained when there is a step in a lower substrate (or a layer) or when a pattern is formed.

Another embodiment relates to a method of synthesizing the aforementioned compound.

The method for synthesizing the aforementioned compound according to an embodiment may include preparing a compound including a cyclopentadienone moiety, and reacting the compound including the cyclopentadienone moiety with a compound having an ethynyl group to obtain the polycyclic aromatic compound.

The preparing of the compound including the cyclopentadienone moiety may include preparing a compound represented by Chemical Formula 12 that will be described below.

In the reacting of the compound including the cyclopentadienone moiety with the compound having the ethynyl group to obtain the polycyclic aromatic compound, the compound including the cyclopentadienone moiety may be a moiety represented by Chemical Formula 12 that will be described below and the compound having the ethynyl group may have at least two ethynyl groups, e.g., an aromatic compound having at least two ethynyl groups.

The reacting of the compound including the cyclopentadienone moiety with the compound having the ethynyl group to obtain the polycyclic aromatic compound may include reacting the compound represented by Chemical Formula 12 with the aromatic compound having at least two ethynyl groups to obtain the polycyclic aromatic compound.

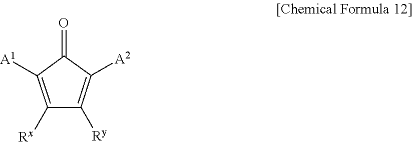

In Chemical Formula 12, A¹ and A² may each independently be or include, e.g., a substituted or unsubstituted phenyl group.

The definitions of R^{x} and R^{y} may be the same as described above.

In an implementation, A¹ and A² may each independently be or include, e.g., an unsubstituted phenyl group; a phenyl group substituted with a substituted or unsubstituted C1 to C30 alkyl group, a substituted or unsubstituted C6 to C30 aryl group, a substituted or unsubstituted C3 to C30 cycloalkyl group, a substituted or unsubstituted C3 to C30 heterocyclic group, or a combination thereof.

In an implementation, A¹ and A² may be the same as or different from each other, e.g. may be the same as each other.

In an implementation, A¹ and A² may be an unsubstituted phenyl group.

In an implementation, A¹, A², R^{x}, and R^{y} may not include a hydroxy group, an amino group, a mercapto group, a substituted or unsubstituted C1 to C30 alkoxy group, a substituted or unsubstituted C1 to C30 alkylamino group, or a substituted or unsubstituted C1 to C30 thioalkoxy group. Accordingly, a decrease in etch resistance may be prevented by maintaining a high carbon content, and specifically, a decrease in etch resistance for the CFx etching gas may be prevented.

In an implementation, the aromatic compound having at least two ethynyl groups may have at least 3 ethynyl groups or at least 4 ethynyl groups, e.g., 2, 3, 4, 5, or 6 ethynyl groups.

In an implementation, the aromatic compound having at least two ethynyl groups may include single aromatic ring or a non-condensed aromatic ring in which two or more aromatic rings are linked by a single bond.

In an implementation, the aromatic compound having at least two ethynyl groups may include a substituted or unsubstituted aromatic ring moiety of Group 1.

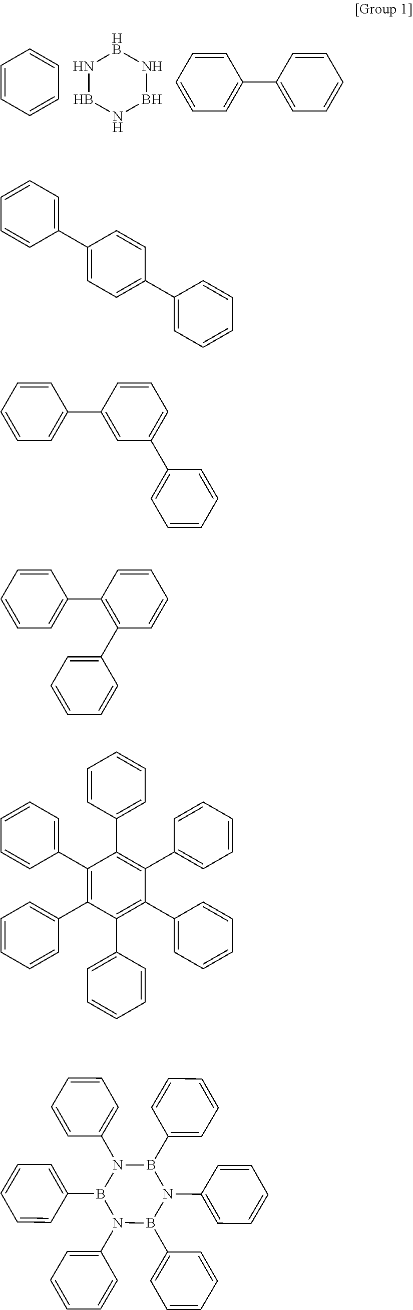

In the definition of Group 1, “substituted” means that the moiety is further substituted with another substituent, and “unsubstituted” means that it is not further substituted with another substituent. In an implementation, the other substituent may include, e.g., the aforementioned substituted or unsubstituted C3 to C20 branched alkyl group.

In an implementation, the aromatic compound having at least two ethynyl groups may include, e.g., a substituted or unsubstituted compound of Group 2.

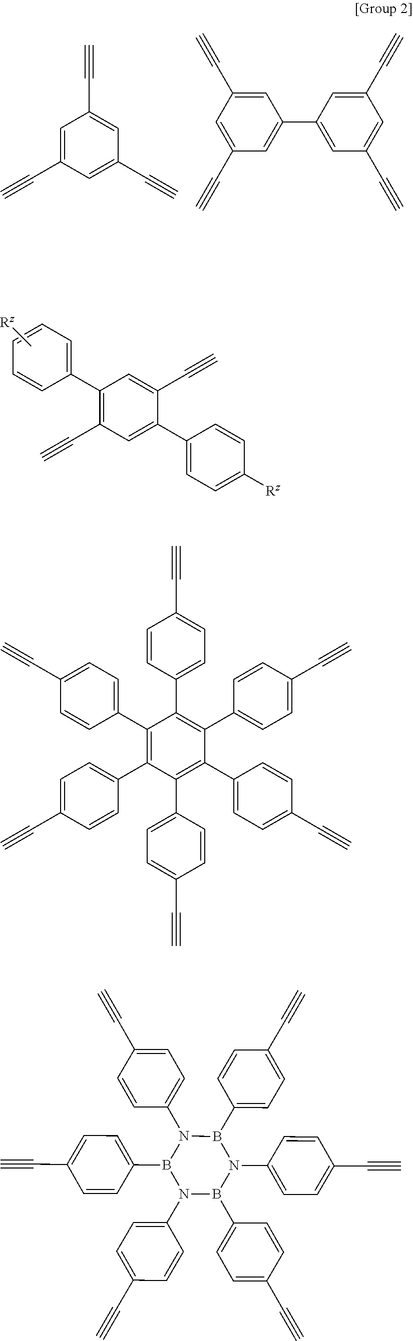

In Group 2, R^{z} may be the same as described above, and in the definition of Group 2, “substituted” means that it is further substituted with another substituent, and “unsubstituted” means that it is not further substituted with another substituent.

The reacting of the compound represented by Chemical Formula 12 with the aromatic compound having at least two ethynyl groups to obtain the polycyclic aromatic compound may include heat-treating, e.g. heat-treating at a temperature of less than or equal to about 300° C., about 50° C. to 300° C., about 70° C. to about 250° C., or about 100° C. to about 200° C.

In an implementation, the reacting of the compound represented by Chemical Formula 12 with the aromatic compound having at least two ethynyl groups to obtain the polycyclic aromatic compound may include obtaining a polycyclic aromatic compound by Diels-Alder reaction and elimination reaction of the aforementioned compound represented by Chemical Formula 12 and the aforementioned aromatic compound having at least two ethynyl groups described above.

In an implementation, in the reacting of the compound represented by Chemical Formula 12 with the aromatic compound having at least two ethynyl groups to obtain the polycyclic aromatic compound, the polycyclic aromatic compound may be a non-condensed polycyclic aromatic compound, e.g., represented by one of Chemical Formulae 7 to 11. In an implementation, the polycyclic aromatic compound may have, e.g., 40 or more carbon atoms, 48 or more carbon atoms, 50 or more carbon atoms, or 54 or more carbon atoms, and 250 or fewer carbon atoms, 230 or fewer carbon atoms, 210 or fewer carbon atoms, 200 or fewer carbon atoms, 180 or fewer carbon atoms, or 150 or fewer carbon atoms.

In an implementation, the method for synthesizing the compound may further include forming a condensed polycyclic aromatic ring having 40 or more carbon atoms by cyclodehydrogenation of the polycyclic aromatic compound, and accordingly, the aforementioned compound represented by Chemical Formulae 2 to 6 may be formed. In an implementation, the condensed polycyclic aromatic ring may have, e.g., 40 or more carbon atoms, 48 or more carbon atoms, 50 or more carbon atoms, or 54 or more carbon atoms, and 250 or fewer carbon atoms, 230 or fewer carbon atoms, 210 or fewer carbon atoms, 200 or fewer carbon atoms, 180 or fewer carbon atoms, or 150 or fewer carbon atoms. In an implementation, the condensed polycyclic aromatic ring having 40 or more carbon atoms may be nanographene. Herein, the definition of the nanographene may be the same as described above.

In an implementation, the forming of the condensed polycyclic aromatic ring having 40 or more carbon atoms by cyclodehydrogenation of the polycyclic aromatic compound may include treating the polycyclic aromatic compound with an acid catalyst. The acid catalyst may be Lewis acid, e.g., an inorganic acid catalyst. In an implementation, the acid catalyst may be, e.g., a metal acid catalyst.

In an implementation, the acid catalyst may include, e.g., iron (III) chloride, 2,3-dichloro-5,6-dicyano-1,4-benzoquinone, ozone, or a combination thereof.

The synthesis method of the aforementioned compound may be beneficial in that a compound including nanographene as a core is synthesized from small molecules (bottom-up), and thus graphene may be easily formed.

According to another embodiment, a hardmask composition including the aforementioned compound and a solvent is provided.

The solvent used in the hardmask composition may be a suitable solvent having sufficient solubility or dispersibility with respect to the compound, e.g., propylene glycol, propylene glycol diacetate, methoxypropanediol, diethylene glycol, diethylene glycolbutylether, tri(ethylene glycol)monomethylether, propylene glycolmonomethylether, propylene glycolmonomethyl ether acetate, cyclohexanone, ethyllactate, gamma-butyrolactone, N,N-dimethyl formamide, N,N-dimethyl acetamide, methylpyrrolidone, methylpyrrolidinone, acetylacetone, or ethyl 3-ethoxypropionate.

The compound may be included in an amount of about 0.1 wt % to about 50 wt %, e.g., about 0.5 wt % to about 40 wt %, about 1 wt % to about 30 wt %, or about 2 wt % to 20 wt %, based on a total weight of the hardmask composition. When the compound is included within the ranges, a thickness, surface roughness and planarization of the hardmask may be controlled.

In an implementation, the hardmask composition may further include an additive, e.g., a surfactant, a cross-linking agent, a thermal acid generator, or a plasticizer.

The surfactant may include, e.g., a fluoroalkyl-based compound, an alkylbenzene sulfonate salt, an alkyl pyridinium salt, polyethylene glycol, or a quaternary ammonium salt.

The cross-linking agent may include, e.g., a melamine-based, substituted urea-based, or a polymer-based cross-linking agent. In an implementation, it may be a cross-linking agent having at least two cross-linking forming substituents, e.g., methoxymethylated glycoluril, butoxymethylated glycoluril, methoxymethylated melamine, butoxymethylated melamine, methoxymethylated benzoguanamine, butoxymethylated benzoguanamine, methoxymethylatedurea, butoxymethylatedurea, methoxymethylated thiourea, butoxymethylated thiourea, or the like.

The cross-linking agent may be a cross-linking agent having high heat resistance. The cross-linking agent having high heat resistance may be a compound including a cross-linking substituent including an aromatic ring (e.g., a benzene ring, or a naphthalene ring) in the molecule.

The thermal acid generator may include, e.g., an acidic compound such as p-toluene sulfonic acid, trifluoromethane sulfonic acid, pyridinium p-toluene sulfonic acid, salicylic acid, sulfosalicylic acid, citric acid, benzoic acid, hydroxybenzoic acid, naphthalene carbonic acid, 2,4,4,6-tetrabromocyclohexadienone, benzointosylate, 2-nitrobenzyltosylate, other organosulfonic acid alkylester, or the like.

The additive may be included in an amount of, e.g., about 0.001 to 40 parts by weight, about 0.01 to 30 parts by weight, or about 0.1 to 20 parts by weight, based on 100 parts by weight of the hardmask composition. Within the ranges, solubility may be improved while optical properties of the hardmask composition are not changed.

According to another embodiment, an organic layer produced using the hardmask composition is provided. The organic layer may be, e.g., formed by coating the hardmask composition on a substrate and heat-treating it for curing and may include, e.g., a hardmask layer, a planarization layer, a sacrificial layer, a filler, and the like for an electronic device.

Hereinafter, a method of forming patterns using the aforementioned hardmask composition is described.

A method of forming patterns according to an embodiment may include forming a material layer on a substrate, applying a hardmask composition including the aforementioned compound and solvent on the material layer, heat-treating the hardmask composition to form a hardmask layer, forming a photoresist layer on the hardmask layer, exposing and developing the photoresist layer to form a photoresist pattern, selectively removing the hardmask layer using the photoresist pattern to expose a portion of the material layer, and etching the exposed portion of the material layer.

The substrate may be, e.g., a silicon wafer, a glass substrate, or a polymer substrate.

The material layer may be a material to be finally patterned, e.g., a metal layer such as an aluminum layer and a copper layer, a semiconductor layer such as a silicon layer, or an insulation layer such as a silicon oxide layer and a silicon nitride layer. The material layer may be formed through a method such as a chemical vapor deposition (CVD) process.

The hardmask composition is the same as described above, and may be applied by spin-on coating in a form of a solution. In an implementation, a thickness of the hardmask composition may be, e.g., about 50 Å to about 200,000 Å.

The heat-treating of the hardmask composition may be performed, e.g., at about 100° C. to about 700° C. for about 10 seconds to about 1 hour.

In an implementation, the method may further include forming a silicon-containing thin layer on the hardmask layer. The silicon-containing thin layer may include, e.g., SiCN, SiOC, SiON, SiOCN, SiC, SiO, SiN, or the like.

In an implementation, the method may further include forming a bottom antireflective coating (BARC) on the silicon-containing thin layer or on the hardmask layer before forming the photoresist layer.

Exposure of the photoresist layer may be performed using, e.g. ArF, KrF, or EUV. After exposure, heat-treating may be performed at about 100° C. to about 700° C.

The etching process of the exposed portion of the material layer may be performed through a dry etching process using an etching gas and the etching gas may be, e.g., CHF₃, CF₄, Cl₂, BCl₃, or a mixed gas thereof.

The etched material layer may be formed in a plurality of patterns, and the plurality of patterns may include a metal pattern, a semiconductor pattern, an insulation pattern, or the like, e.g., diverse patterns of a semiconductor integrated circuit device.

Hereinafter, the embodiments are illustrated in more detail with reference to examples. However, these examples are exemplary, and the present scope is not limited thereto.

### SYNTHESIS OF COMPOUNDS

### Synthesis Example 1

After putting an agitator in a 1 L round-bottomed flask dried in a vacuum oven, 40.3 g of 1,2-bis(3,5-di-tert-butylphenyl)ethyne (molecular weight: 402.67) and 12.7 g of iodine were put therein, 0.5 L of N,N′-dimethylsulfoxide was put therein, and after a condenser was connected thereto, the mixture was refluxed for 6 hours. Subsequently, the obtained mixture was sequentially diluted with 0.5 L of a 1 M sodium thiosulfate aqueous solution and 1 L of ethyl acetate and then, extracted with distilled water (three times in total using 1 L each time). An organic layer therefrom was dried with anhydrous sodium sulfate, filtered, and concentrated to obtain 1,2-bis(3,5-di-tertiarybutylphenyl)ethane-1,2-dion (39.5 g). The 1,2-bis(3,5-di-tertiarybutylphenyl)ethane-1,2-dion is represented by Chemical Formula 1a. (a yield: 91%, a molecular weight: 434.66, ¹H NMR (300 MHz, CDCl₃) δ (ppm): 7.82 (d, ⁴J_{HH}=1.8 Hz, 4H), 7.74 (t, ⁴J_{HH}=1.8 Hz, 2H), 1.34 (s, 36H))

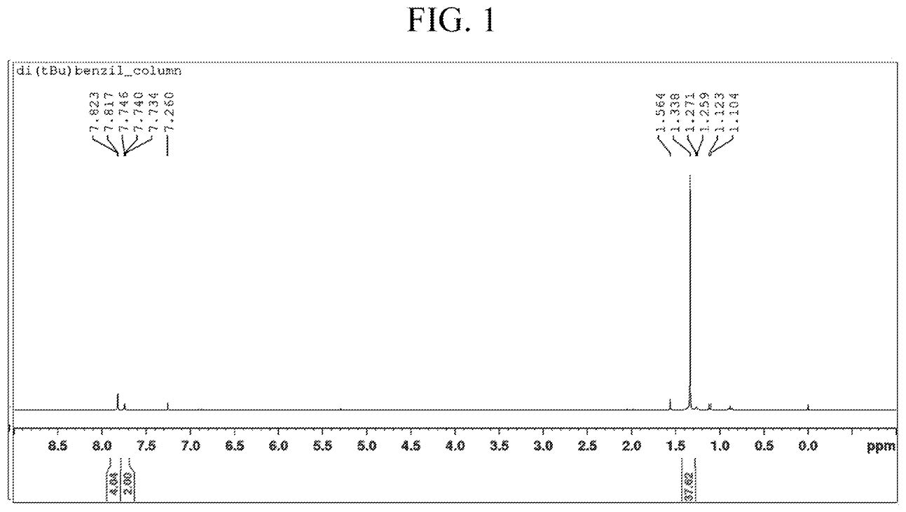

FIG. 1 is a ¹H NMR spectrum of the compound represented by Chemical Formula 1a.

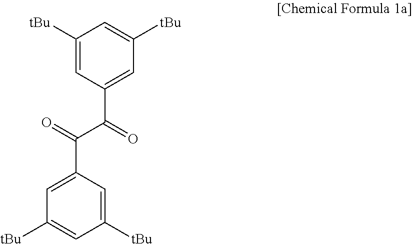 [SMILES: CC(C)(C)c1cc(C(=O)C(=O)c2cc(C(C)(C)C)cc(C(C)(C)C)c2)cc(C(C)(C)C)c1]

### Synthesis Example 2

After putting a stirrer in a 1 L round-bottomed flask dried in a vacuum oven and equipped with a dropping funnel and a condenser, the compound represented by Chemical Formula 1a (39.5 g) according to Synthesis Example 1, 1,3-diphenyl-2-propanone (20.1 g, molecular weight: 210.28), and 700 mL of methanol were put therein and then, refluxed and stirred for 1 hour. Subsequently, a solution prepared by dissolving potassium hydroxide (10.7 g) in 100 mL of methanol was added thereto in a dropwise fashion through the dropping funnel for 30 minutes and then, refluxed and stirred for 50 minutes. The mixture was cooled down to ambient temperature, and then, red precipitates produced therein were filtered, washed with cold ethanol (three times in total using 300 mL each time), and dried to obtain 3,4-bis(3,5-di-tertiarybutylphenyl)-2,5-diphenylcyclopenta-2,4-dien-1-one (40.0 g). The 3,4-bis(3,5-di-tertiarybutylphenyl)-2,5-diphenylcyclopenta-2,4-dien-1-one is represented by Chemical Formula 1b. (a yield: 72%, a molecular weight: 608.91, ¹H NMR (300 MHz, CDCl₃) δ (ppm): 7.15-7.35 (m, 12H), 6.72 (d, ⁴J_{HH}=1.8 Hz, 4H), 1.05 (s, 36H)).

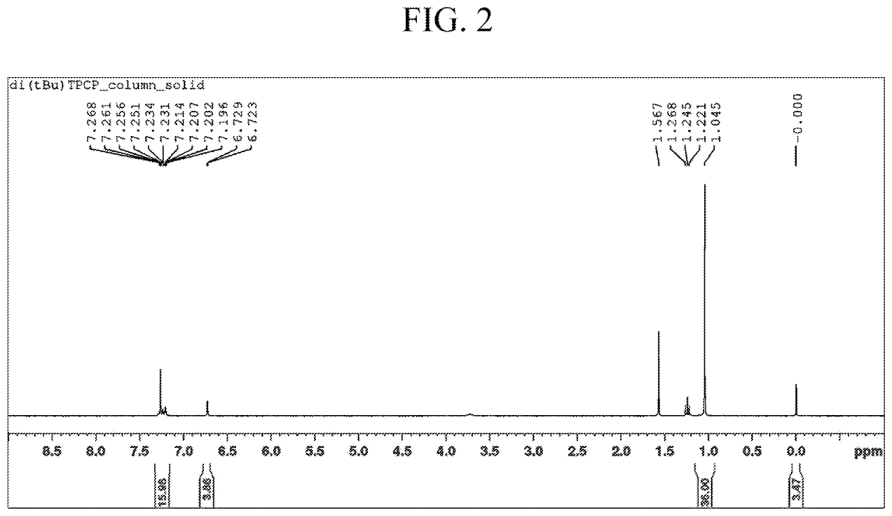

FIG. 2 is a ¹H NMR spectrum of the compound represented by Chemical Formula 1b.

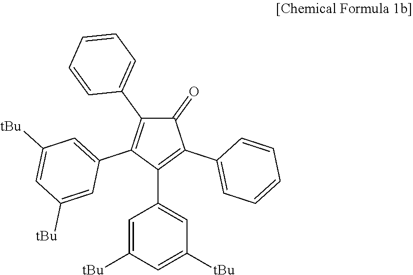 [SMILES: CC(C)(C)c1cc(C2=C(c3ccccc3)C(=O)C(c3ccccc3)=C2c2cc(C(C)(C)C)cc(C(C)(C)C)c2)cc(C(C)(C)C)c1]

### Synthesis Example 3

After putting a stirrer in a 250 mL round-bottomed flask equipped with a condenser, 1,3,5-triethynylbenzene (molecular weight: 150.18, 1.64 g), the compound represented by Chemical Formula 1b (20.0 g) according to Synthesis Example 2, and 100 mL of PGMEA were put therein and then, stirred for a reaction at 150° C. for 24 hours. Subsequently, the mixture was cooled down to ambient temperature and concentrated to obtain the equivalent of a compound represented by Chemical Formula 1c (20.7 g). (a molecular weight: 1892.88, MALDI-TOF [M+H]⁺=1892.5154 m/z)

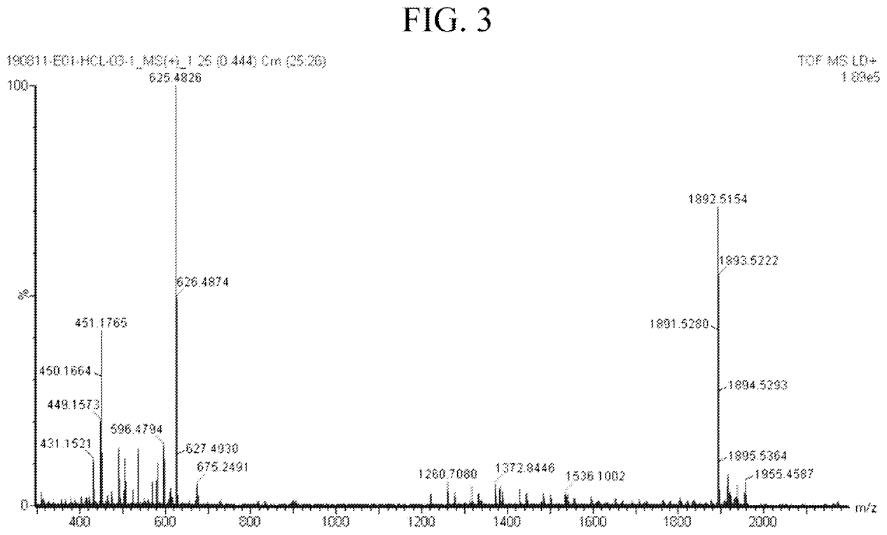

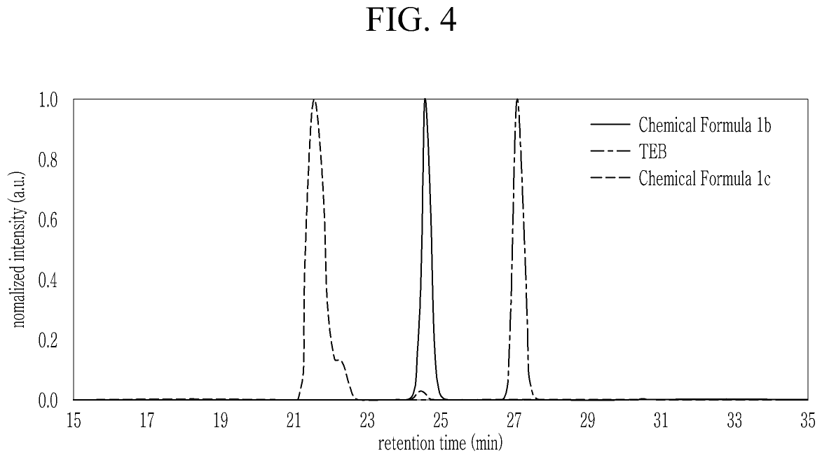

FIG. 3 is a MALDI-TOF mass spectrum of the compound represented by Chemical Formula 1c, FIG. 4 is gel permeation chromatography (GPC) spectra of 1,3,5-triethynylbenzene (TEB) and the compounds represented by Chemical Formulas 1b and 1c.

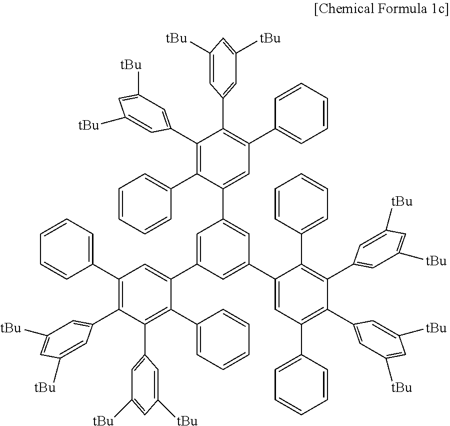 [SMILES: CC(C)(C)c1cc(-c2c(-c3ccccc3)cc(-c3cc(-c4cc(-c5ccccc5)c(-c5cc(C(C)(C)C)cc(C(C)(C)C)c5)c(-c5cc(C(C)(C)C)cc(C(C)(C)C)c5)c4-c4ccccc4)cc(-c4cc(-c5ccccc5)c(-c5cc(C(C)(C)C)cc(C(C)(C)C)c5)c(-c5cc(C(C)(C)C)cc(C(C)(C)C)c5)c4-c4ccccc4)c3)c(-c3ccccc3)c2-c2cc(C(C)(C)C)cc(C(C)(C)C)c2)cc(C(C)(C)C)c1]

### Synthesis Example 4

After putting a stirrer in a 250 mL round-bottomed flask equipped with a condenser, 3,3′,5′,5′-tetraethynylbiphenyl (2.06 g, molecular weight: 250.30), the compound represented by Chemical Formula 1b (20.0 g) according to Synthesis Example 2, and 100 mL of PGMEA were put therein and then, stirred for a reaction at 150° C. for 24 hours. Subsequently, the mixture was cooled down to ambient temperature and concentrated to obtain the equivalent of the compound represented by Chemical Formula 1d (21.1 g). (a molecular weight: 2573.90, MALDI-TOF [M+H]⁺=2574.0427 m/z)

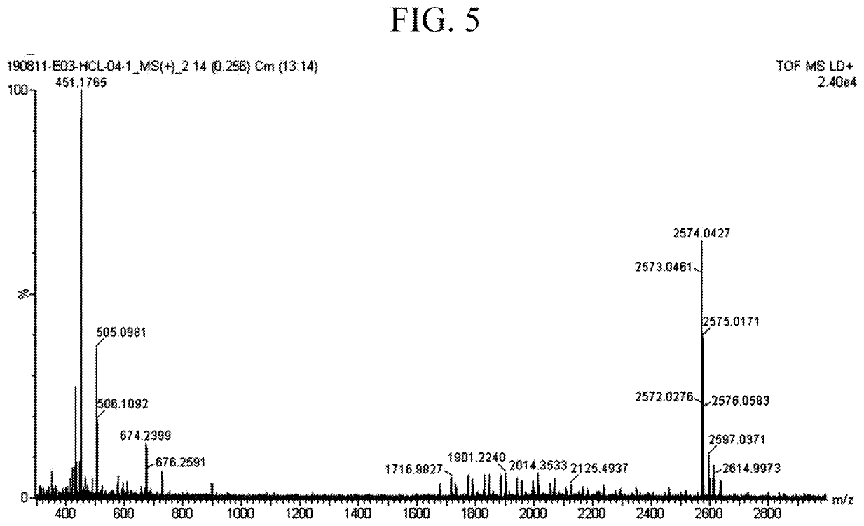

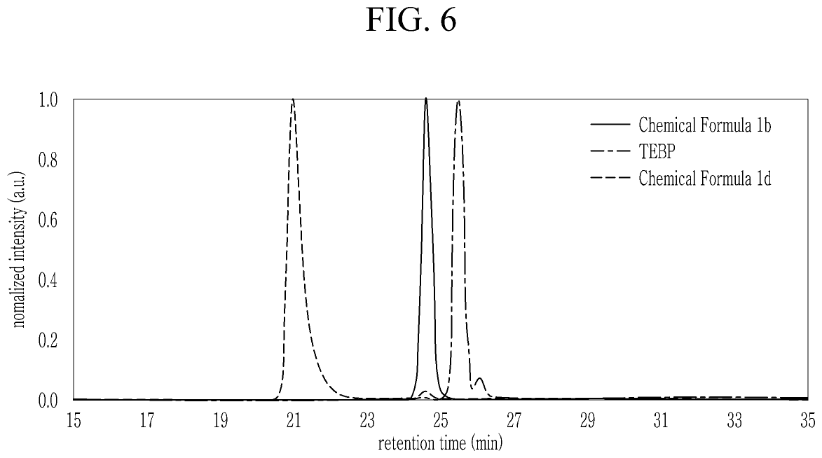

FIG. 5 is a MALDI-TOF mass spectrum of the compound represented by Chemical Formula 1d, and FIG. 6 is gel permeation chromatography (GPC) spectra of 3,3′,5′,5′-tetraethynylbiphenyl (TEBP) and the compounds represented by Chemical Formulas 1b and 1d.

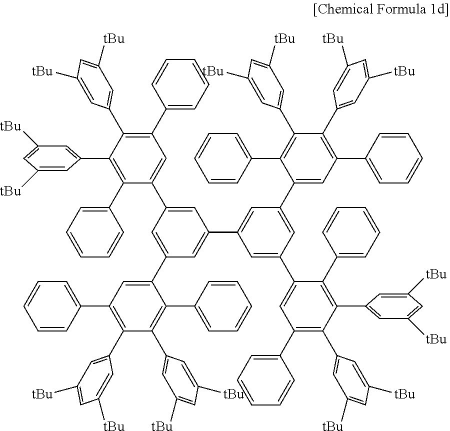 [SMILES: CC(C)(C)c1cc(-c2c(-c3ccccc3)cc(-c3cc(-c4cc(-c5cc(-c6ccccc6)c(-c6cc(C(C)(C)C)cc(C(C)(C)C)c6)c(-c6cc(C(C)(C)C)cc(C(C)(C)C)c6)c5-c5ccccc5)cc(-c5cc(-c6ccccc6)c(-c6cc(C(C)(C)C)cc(C(C)(C)C)c6)c(-c6cc(C(C)(C)C)cc(C(C)(C)C)c6)c5-c5ccccc5)c4)cc(-c4cc(-c5ccccc5)c(-c5cc(C(C)(C)C)cc(C(C)(C)C)c5)c(-c5cc(C(C)(C)C)cc(C(C)(C)C)c5)c4-c4ccccc4)c3)c(-c3ccccc3)c2-c2cc(C(C)(C)C)cc(C(C)(C)C)c2)cc(C(C)(C)C)c1]

### Synthesis Example 5

After putting a stirrer in a 1 L round-bottomed flask dried in a vacuum oven, the compound represented by Chemical Formula 1c (10.0 g) according to Synthesis Example 3 and 500 mL of dichloromethane were put therein, and the flask was put in an ice-bath and stirred. Subsequently, a solution prepared by mixing iron (III) chloride (25.7 g) and nitromethane (200 mL) was put in a dropping funnel and then, added to the flask in a dropwise fashion for 30 minutes, while stirring. After the addition in a dropwise fashion, the ice-bath was removed, and the mixture was additionally stirred for 2 hours. Subsequently, 100 mL of methanol and 100 mL of water were added thereto and then, stirred and concentrated. The concentrated mixture was diluted with 1 L of ethyl acetate, washed with a 0.5 N hydrogen chloride (HCl) aqueous solution (twice, using 1 L each time), washed with water (five times, using 1 L each time), and concentrated to obtain the equivalent of a compound represented by Chemical Formula 1e (9.9 g). (a molecular weight: 1874.74, MALDI-TOF [M+H]⁺=1868.2833 m/z)

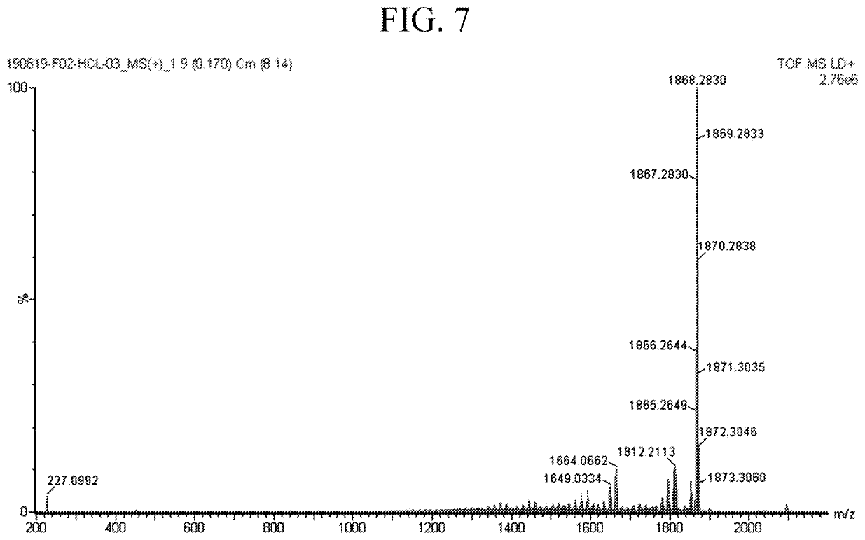

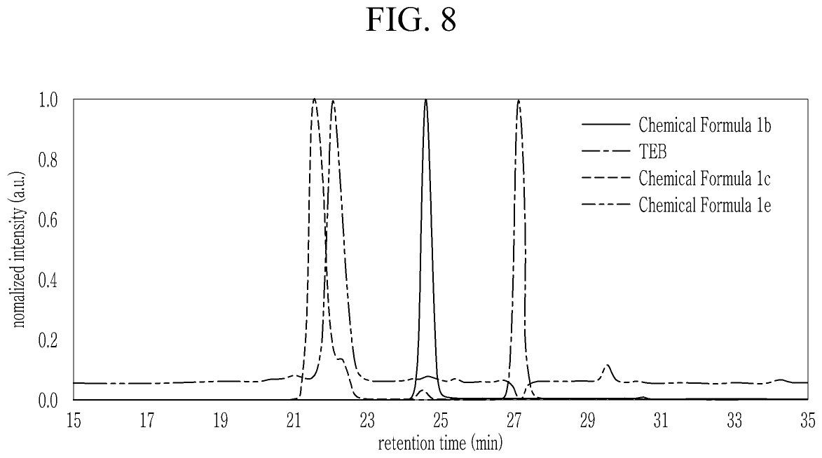

FIG. 7 is a MALDI-TOF mass spectrum of the compound of the compound represented by Chemical Formula 1e, and FIG. 8 is gel permeation chromatography (GPC) spectra of 1,3,5-triethynylbenzene (TEB) and the compounds represented by Chemical Formulas 1b, 1c, and 1e.

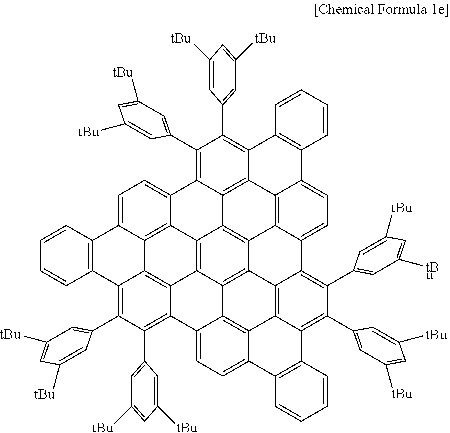 [SMILES: CC(C)(C)c1cc(-c2c(-c3cc(C(C)(C)C)cc(C(C)(C)C)c3)c3c4ccc5c6ccccc6c6c(-c7cc(C(C)(C)C)cc(C(C)(C)C)c7)c(-c7cc(C(C)(C)C)cc(C(C)(C)C)c7)c7c8ccc9c%10ccccc%10c%10c(-c%11cc(C(C)(C)C)cc(C(C)(C)C)c%11)c(-c%11cc(C(C)(C)C)cc(C(C)(C)C)c%11)c%11c%12ccc%13c%14ccccc%14c2c2c%13c%12c%12c(c32)c2c4c5c6c7c2c2c8c9c%10c%11c%122)cc(C(C)(C)C)c1]

### Synthesis Example 6

In a 1 L round-bottomed flask dried in a vacuum oven, the compound represented by Chemical Formula 1d (10.0 g) according to Synthesis Example 4 and dichloromethane (500 mL) were put, and the flask was put in an ice-bath and stirred. Subsequently, a solution prepared by using iron (III) chloride (31.5 g) and nitromethane (200 mL) was put in a dropping funnel and then, added to the flask in a dropwise fashion for 30 minutes, while stirring. After the addition in a dropwise fashion, the ice-bath was removed, and the mixture was additionally stirred for 2 hours. Subsequently, 100 mL of methanol and 100 mL of water were added to the mixture and then, stirred and concentrated. The concentrate mixture was diluted with 1 L of ethyl acetate, washed with a 0.5 N hydrogen chloride (HCl) aqueous solution (twice, using 1 L each time), washed with water (five times, using 1 L each time), and concentrated to obtain a compound represented by Chemical Formula 1f (9.8 g). (MALDI-TOF [M+H]⁺=2479.6055 m/z)

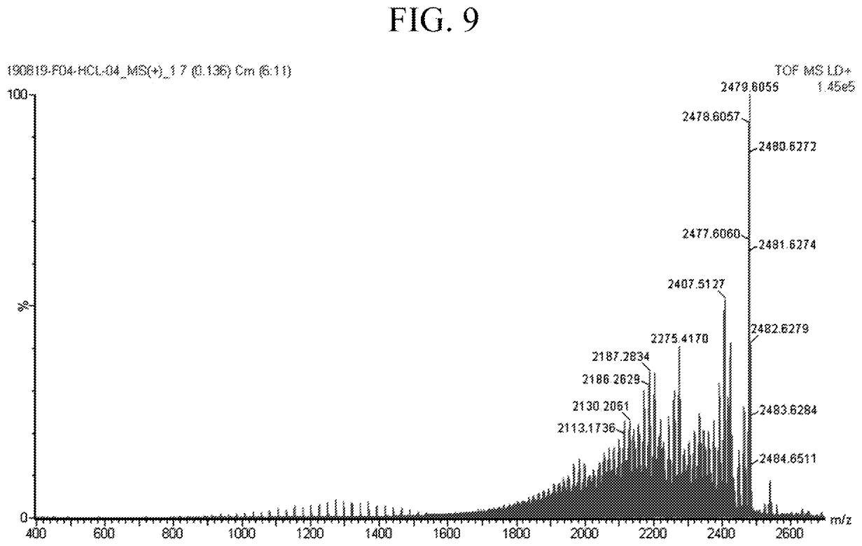

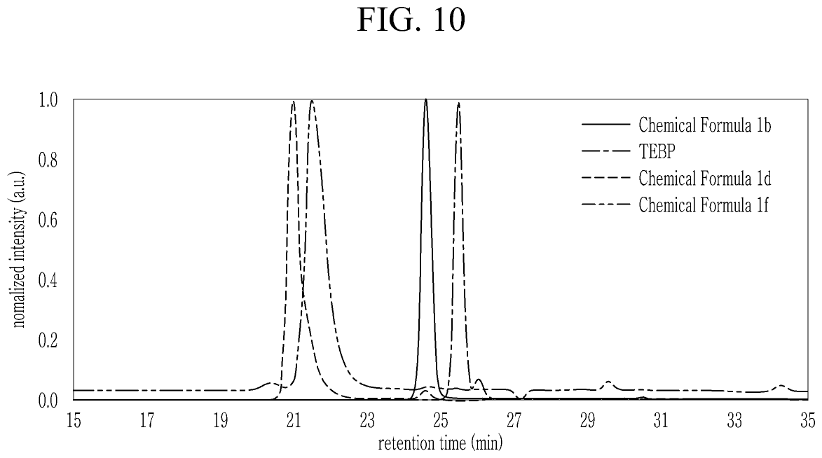

FIG. 9 is a MALDI-TOF mass spectrum of the compound represented by Chemical Formula 1f, and FIG. 10 is gel permeation chromatography (GPC) spectra of 3,3′,5′,5′-tetraethynylbiphenyl (TEBP) and the compounds represented by Chemical Formulas 1b, 1d, and 1f.

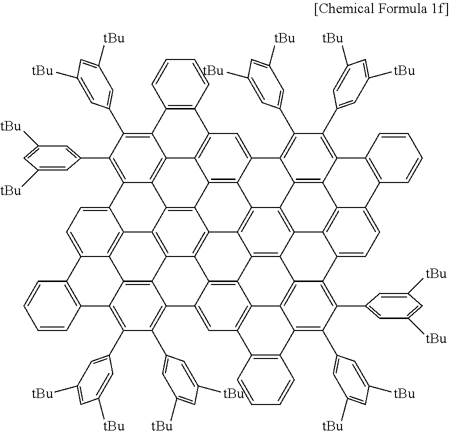 [SMILES: CC(C)(C)c1cc(-c2c(-c3cc(C(C)(C)C)cc(C(C)(C)C)c3)c3c4cc5c6ccccc6c6c(-c7cc(C(C)(C)C)cc(C(C)(C)C)c7)c(-c7cc(C(C)(C)C)cc(C(C)(C)C)c7)c7c8ccc9c%10ccccc%10c%10c(-c%11cc(C(C)(C)C)cc(C(C)(C)C)c%11)c(-c%11cc(C(C)(C)C)cc(C(C)(C)C)c%11)c%11c%12cc%13c%14ccccc%14c%14c(-c%15cc(C(C)(C)C)cc(C(C)(C)C)c%15)c(-c%15cc(C(C)(C)C)cc(C(C)(C)C)c%15)c%15c%16ccc%17c%18ccccc%18c2c2c%17c%16c%16c(c32)c2c4c3c5c6c7c4c5c8c9c%10c%11c5c5c%12c6c%13c%14c%15c%16c6c2c5c34)cc(C(C)(C)C)c1]

### Comparison Synthesis Example 1

In a 500 mL 2-necked round-bottomed flask equipped with a dropping funnel and a condenser, 1-hydroxypyrene (21.8 g, 0.10 mol), 1-naphthol (14.5 g, 0.10 mol), paraformaldehyde (6.0 g, 0.2 mol), diethyl sulfate (15.4 g, 0.10 mol), and propylene glycol monomethyl ether acetate (PGMEA, 115 g) were put and then, stirred at 110° C. for 5 hours to 48 hours to perform a polymerization reaction, and when a weight average molecular weight reached 1,000 to 1,500, the reaction was completed. After completing the polymerization reaction, the resultant was slowly cooled down to ambient temperature, diluted with 500 ml of ethyl acetate, and washed ten times with 500 ml of distilled water. An organic layer therefrom was concentrated under a reduced pressure, diluted with tetrahydrofuran (THF, 200 ml), and slowly added in a dropwise fashion to 1.5 L of hexane to obtain precipitates. The precipitates were filtered, dissolved again in tetrahydrofuran 200 ml, and then, slowly added in a dropwise fashion to 1.5 L of hexane to obtain precipitates again. The precipitates were filtered and dried to obtain a polymer including a structural unit represented by Chemical Formula A. (Mw: 1,500)

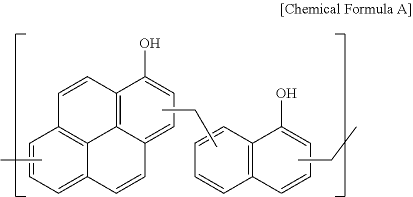

### Comparison Synthesis Example 2

A polymer including a structural unit represented by Chemical Formula B was obtained according to the same method as Comparison Synthesis Example 1 except that 4,4′-(9H-fluoren-9-ylidene)bisphenol (35.0 g, 0.10 mol), 1,4-bis(methoxymethyl)benzene (16.6 g, 0.10 mol), diethyl sulfate (15.4 g, 0.10 mol), and 134 g of PGMEA were used instead of 1-hydroxypyrene (21.8 g, 0.10 mol), 1-naphthol (14.5 g, 0.10 mol), paraformaldehyde (6.0 g, 0.2 mol), diethyl sulfate (15.4 g, 0.10 mol), and 115 g of PGMEA. (Mw: 1,700)

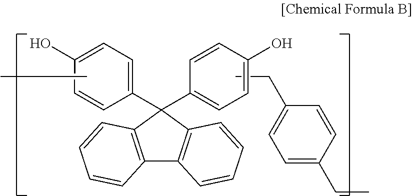

Preparation of Hardmask Composition

The compounds and polymers according to Synthesis Examples 3 to 6 and

Comparison Synthesis Examples 1 and 2 were respectively uniformly dissolved in an appropriate solvent of PGMEA, cyclohexanone, or PGMEA/cyclohexanone mixed in a volume ratio of 1:10 to 10:1, to prepare each hardmask composition at a concentration of 15 wt % according to Examples 1 to 4 and Comparative Examples 1 and 2.

Evaluation: Etch Resistance

The hardmask compositions according to Examples 1 to 4 and Comparative Examples 1 and 2 were respectively spin-coated on a silicon wafer, heat-treated on a hot plate at about 530° C. for 2 minutes to form about 3,000 to 4,000 Å-thick organic films. Subsequently, the organic films were dry-etched by using CFx mixed gas (100 mT/600 W/42CF₄/18CHF₃/600 Ar/15O₂) for 100 seconds, and then, thicknesses thereof were measured again.

A thickness difference of the organic layers before and after the dry etching and etching time were used to calculate a bulk etch rate (BER) according to Calculation Equation 1.

Etch rate (Å/s)=(Initial thickness of organic layer−Thickness of organic layer after etching)/Etching time [Calculation Equation 1]

The results are shown in Table 1.

TABLE 1

| | CFₓ Bulk etch rate |
|---|---|
| | (Å/s) |
| Example 1 (Synthesis Example 3) | 24.8 |
| Example 2 (Synthesis Example 4) | 24.4 |
| Example 3 (Synthesis Example 5) | 19.3 |
| Example 4 (Synthesis Example 6) | 18.8 |
| Comparative Example 1 (Comparative Synthesis | 29.6 |
| Example 1) | |
| Comparative Example 2 (Comparative Synthesis | 27.3 |
| Example 2) | |

The organic films according to Examples 1 to 4 exhibited sufficient etch resistance against the etching gas and thus improved etch resistance, compared with the organic films according to Comparative Examples 1 and 2.

By way of summation and review, according to small-sizing the pattern to be formed, it may be difficult to provide a fine pattern having an excellent profile by using some lithographic techniques. Accordingly, an auxiliary layer, called a hardmask layer, may be formed between the material layer and the photoresist layer to provide a fine pattern.

One or more embodiments may provide a compound that may be effectively applied to a hardmask layer.

According to an embodiment, etch resistance of the hardmask layer may be ensured.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound comprising a condensed or non-condensed polycyclic aromatic core having 40 or more carbon atoms and a plurality of substituents at a periphery of the core,
wherein the plurality of substituents are each independently a substituted or unsubstituted C3 to C20 branched alkyl group, a C6 to C30 aryl group substituted with a substituted or unsubstituted C3 to C20 branched alkyl group, a C3 to C30 cycloalkyl group substituted with a substituted or unsubstituted C3 to C20 branched alkyl group, a C3 to C30 heterocyclic group substituted with a substituted or unsubstituted C3 to C20 branched alkyl group, or a combination thereof.

2. The compound as claimed in claim 1, wherein:
the plurality of substituents includes a C6 to C30 aryl group substituted with a substituted or unsubstituted C3 to C20 branched alkyl group, and
the C6 to C30 aryl group substituted with a substituted or unsubstituted C3 to C20 branched alkyl group is a phenyl group substituted with at least two substituted or unsubstituted C3 to C20 branched alkyl groups, a naphthyl group substituted with at least two substituted or unsubstituted C3 to C20 branched alkyl groups, an anthracenyl group substituted with at least two substituted or unsubstituted C3 to C20 branched alkyl groups, a phenanthrenyl group substituted with at least two substituted or unsubstituted C3 to C20 branched alkyl groups, a pyrenyl group substituted with at least two substituted or unsubstituted C3 to C20 branched alkyl groups, or a combination thereof.

3. The compound as claimed in claim 1, wherein two substituents of the plurality of substituents are bonded to one aromatic ring of the condensed or non-condensed polycyclic aromatic core at ortho positions of the one aromatic ring.

4. The compound as claimed in claim 1, wherein:
the plurality of substituents includes a C6 to C30 aryl group substituted with a substituted or unsubstituted C3 to C20 branched alkyl group,
the C6 to C30 aryl group substituted with a substituted or unsubstituted C3 to C20 branched alkyl group is a group represented by Chemical Formula 1: 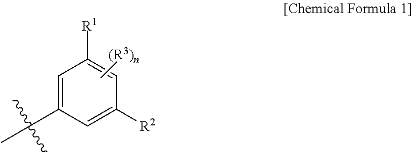
in Chemical Formula 1,
R¹ and R² are independently a substituted or unsubstituted C3 to C20 branched alkyl group,
R³ is independently a substituted or unsubstituted C1 to C30 alkyl group, a substituted or unsubstituted C2 to C30 alkenyl group, a substituted or unsubstituted C2 to C30 alkynyl group, a substituted or unsubstituted C6 to C30 aryl group, a substituted or unsubstituted C3 to C30 heterocyclic group, a halogen, a cyano group, or a combination thereof,
n is an integer of 0 to 3, and 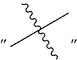
is a linking point with the condensed or non-condensed polycyclic aromatic core.

5. The compound as claimed in claim 1, wherein the substituted or unsubstituted C3 to C20 branched alkyl group is a substituted or unsubstituted C3 to C20 iso-alkyl group, a substituted or unsubstituted C4 to C20 sec-alkyl group, a substituted or unsubstituted C4 to C20 tert-alkyl group, or a substituted or unsubstituted C5 to C20 neo-alkyl group.

6. The compound as claimed in claim 1, wherein the substituted or unsubstituted C3 to C20 branched alkyl group is a substituted or unsubstituted iso-propyl group, a substituted or unsubstituted iso-butyl group, a substituted or unsubstituted sec-butyl group, a substituted or unsubstituted tert-butyl group, a substituted or unsubstituted iso-pentyl group, a substituted or unsubstituted sec-pentyl group, a substituted or unsubstituted tert-pentyl group, or a substituted or unsubstituted neo-pentyl group.

7. The compound as claimed in claim 1, wherein the condensed or non-condensed polycyclic aromatic core includes a borazine moiety.

8. The compound as claimed in claim 1, wherein:
the condensed or non-condensed polycyclic aromatic core is a condensed polycyclic aromatic ring, and
the condensed polycyclic aromatic ring has a nanographene structure.

9. The compound as claimed in claim 8, wherein a number of the substituents relative to a total number of substitutable positions at the periphery of the condensed polycyclic aromatic core is greater than or equal to about 10%.

10. The compound as claimed in claim 8, wherein the condensed polycyclic aromatic core is a particle having a size of about 1 nm to about 20 nm.

11. The compound as claimed in claim 8, wherein:
the compound is represented by one of Chemical Formulae 2 to 6: 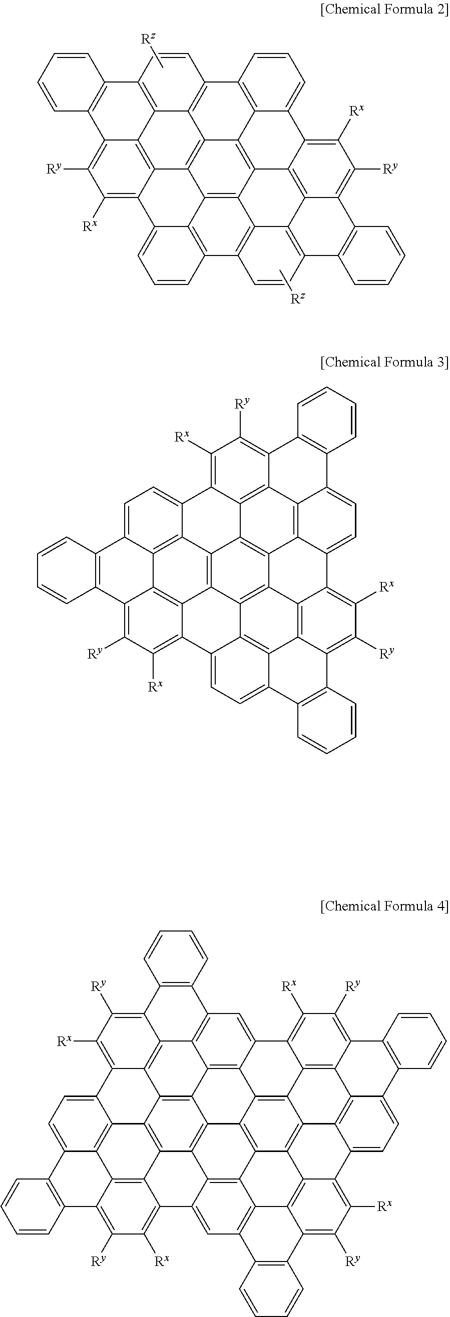 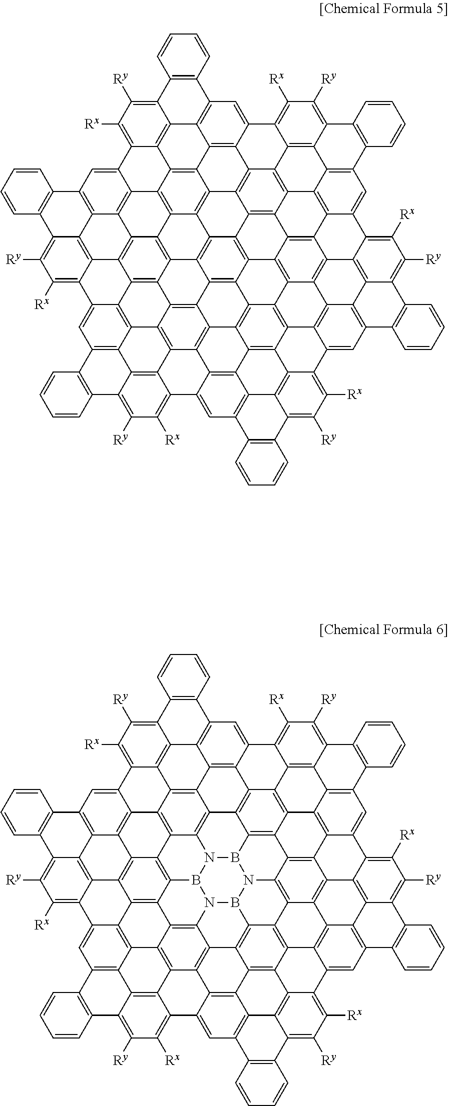
in Chemical Formulae 2 to 6, R^{x} to R^{z} are independently a substituted or unsubstituted C3 to C20 branched alkyl group, a C6 to C30 aryl group substituted with a substituted or unsubstituted C3 to C20 branched alkyl group, a C3 to C30 cycloalkyl group substituted with a substituted or unsubstituted C3 to C20 branched alkyl group, or a C3 to C30 heterocyclic group substituted with a substituted or unsubstituted C3 to C20 branched alkyl group.

12. The compound as claimed in claim 11, wherein:
R^{x} and R^{y} are independently a group represented by Chemical Formula 1: 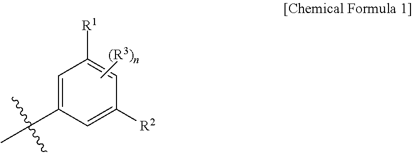
in Chemical Formula 1,
R¹ and R² are independently a substituted or unsubstituted C3 to C20 branched alkyl group,
R³ is a substituted or unsubstituted C1 to C30 alkyl group, a substituted or unsubstituted C2 to C30 alkenyl group, a substituted or unsubstituted C2 to C30 alkynyl group, a substituted or unsubstituted C6 to C30 aryl group, a substituted or unsubstituted C3 to C30 heterocyclic group, or a combination thereof,
n is an integer of 0 to 3, and 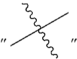
is a linking point with the nanographene structure.

13. The compound as claimed in claim 11, wherein R^{z} is a substituted or unsubstituted C3 to C20 branched alkyl group.

14. The compound as claimed in claim 1, wherein:
the condensed or non-condensed polycyclic aromatic core is a non-condensed polycyclic aromatic core, and
the compound is represented by one of Chemical Formulae 7 to 11: 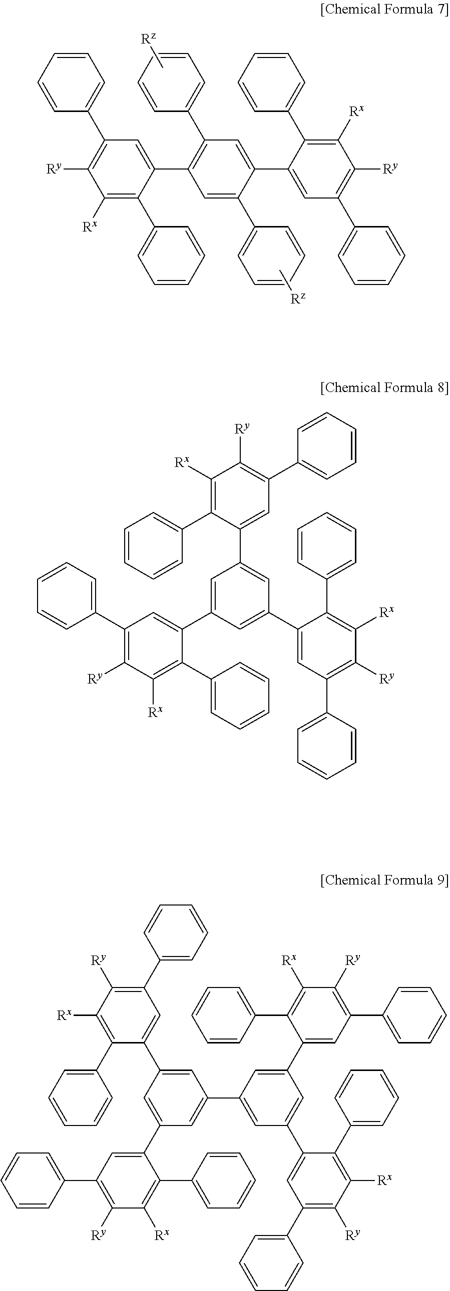 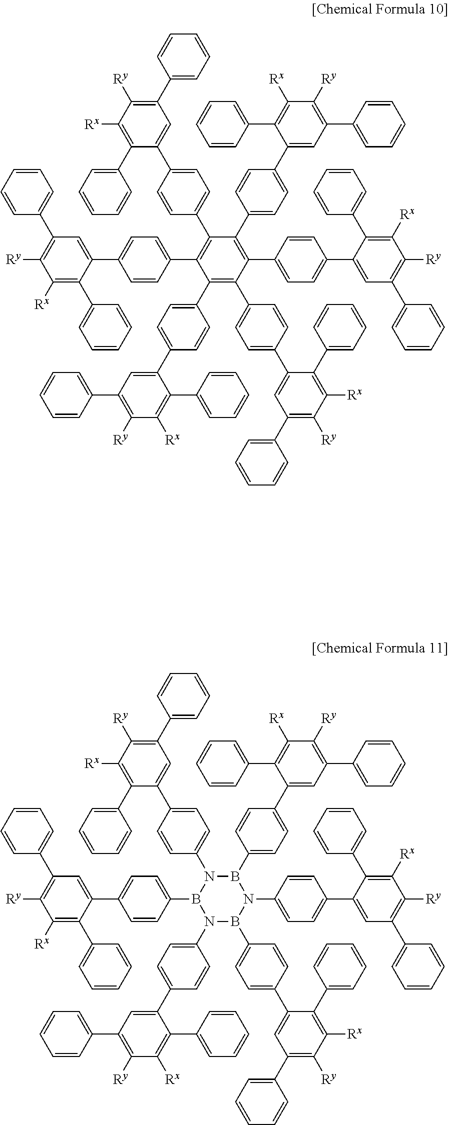
in Chemical Formulae 7 to 11, R^{x} to R^{z} are independently a substituted or unsubstituted C3 to C20 branched alkyl group, a C6 to C30 aryl group substituted with a substituted or unsubstituted C3 to C20 branched alkyl group, a C3 to C30 cycloalkyl group substituted with a substituted or unsubstituted C3 to C20 branched alkyl group, or a C3 to C30 heterocyclic group substituted with a substituted or unsubstituted C3 to C20 branched alkyl group.

15. The compound as claimed in claim 14, wherein:
R^{x} and R^{y} are independently a group represented by Chemical Formula 1: 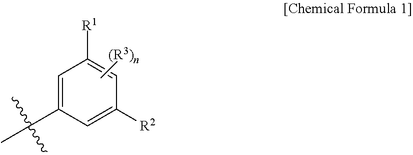
in Chemical Formula 1,
R¹ and R² are independently a substituted or unsubstituted C3 to C20 branched alkyl group,
R³ is a substituted or unsubstituted C1 to C30 alkyl group, a substituted or unsubstituted C2 to C30 alkenyl group, a substituted or unsubstituted C2 to C30 alkynyl group, a substituted or unsubstituted C6 to C30 aryl group, a substituted or unsubstituted C3 to C30 heterocyclic group, or a combination thereof,
n is an integer of 0 to 3, and 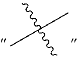
is a linking point with the non-condensed polycyclic aromatic core.

16. A method of synthesizing a compound, the method comprising:
preparing a compound represented by Chemical Formula 12, and
reacting the compound represented by Chemical Formula 12 with an aromatic compound having at least two ethynyl groups to obtain a polycyclic aromatic compound, 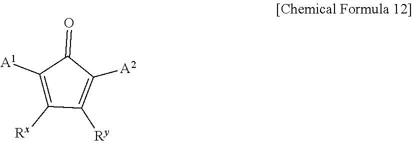
wherein, in Chemical Formula 12,
A¹ and A² are independently a substituted or unsubstituted phenyl group, and
R^{x} and R^{y} are independently a substituted or unsubstituted C3 to C20 branched alkyl group, a C6 to C30 aryl group substituted with a substituted or unsubstituted C3 to C20 branched alkyl group, a C3 to C30 cycloalkyl group substituted with a substituted or unsubstituted C3 to C20 branched alkyl group, or a C3 to C30 heterocyclic group substituted with a substituted or unsubstituted C3 to C20 branched alkyl group.

17. The method as claimed in claim 16, wherein the aromatic compound having at least two ethynyl groups includes a single aromatic ring or a non-condensed aromatic ring structure in which two or more aromatic rings are linked by a single bond.

18. The method as claimed in claim 16, further comprising forming a condensed polycyclic aromatic ring having 40 or more carbon atoms by cyclodehydrogenation of the polycyclic aromatic compound,
wherein the condensed polycyclic aromatic ring having 40 or more carbon atoms has a nanographene structure.

19. A hardmask composition, comprising:
the compound as claimed in claim 1; and
a solvent.

20. A method of forming patterns, the method comprising:
applying the hardmask composition as claimed in claim 19 on a material layer and heat treating the hardmask composition to form a hardmask layer,
forming a photoresist layer on the hardmask layer,
exposing and developing the photoresist layer to form a photoresist pattern,
selectively removing the hardmask layer using the photoresist pattern to expose a portion of the material layer, and
etching an exposed portion of the material layer.
